(19) United States
(12) Patent Application Publication
(10) Pub. No.: US 2023/0218752 A1
(43) Pub. Date: Jul. 13, 2023

(54) POLYMERIC NANOPARTICLES AS VACCINE ADJUVANTS

(71) Applicant: THE PROVOST, FELLOWS, SCHOLARS AND OTHER MEMBERS OF THE BOARD OF TRINITY COLLEGE DUBLIN, Dublin (IE)

(72) Inventors: Ed LAVELLE, Dublin (IE); Natalia MUNOZ-WOLF, Dublin (IE)

(21) Appl. No.: 17/787,051

(22) PCT Filed: Dec. 21, 2020
(86) PCT No.: PCT/EP2020/087456

§ 371 Date: Jun. 17, 2022

(30) Foreign Application Priority Data
Dec. 20, 2019 (GB) 1918963.8

(51) Int. Cl. A61K 39/39; A61K 39/215; A61K 39/145; A61K 39/29; A61K 39/25; A61K 39/12; A61P 37/04

(52) U.S. Cl. CPC A61K 39/39; A61K 39/12; A61K 39/25; A61K 39/29; A61K 39/145; A61K 39/215; A61P 37/04; A61K 2039/55555

(57) ABSTRACT

Provided by the current invention is a biocompatible polymeric particle having a diameter of from 50nm to 65nm and methods for use thereof. A method to induce antigen specific CD8 T cells and Thl response is also provided.

### FIELD OF THE INVENTION

The invention relates to polymeric particles. In particular, the invention relates to biocompatible and/or biodegradable polymeric nanoparticles having a diameter of from 50nm ± 10nm and methods for use thereof. Notably, the invention relates to biocompatible and/or biodegradable polymeric nanoparticles having a diameter of from 50nm ± 10nm as an adjuvant for induction of cell mediated immunity in addition to antibody responses. The invention also relates to polymeric nanoparticles having a diameter of 50nm±10nm that can stimulate caspase-11 and/or Gasdermin D dependent cellular immunity to an antigen.

### BACKGROUND TO THE INVENTION

Vaccines are one of the most effective ways to prevent infectious disease and the spread of microbes that can cause disease. A vaccine is a preparation that provides a subject with acquired immunity to a disease, such that the disease is prevented in the subject, or only mild symptoms are experienced.

Traditionally, vaccines are made using small amounts of weak or dead microorganisms, for example viruses or bacteria that cause disease. As vaccine research evolved, it was discovered that it was not necessary to use the whole microorganism to provide protection and that, instead, small components of the microorganism, called antigens, could be used. Vaccines comprising purified antigens cause less adverse reactions and are safer than vaccines made of whole microorganisms. However, highly purified antigens are often too weak to activate the immune system in a subject. To solve this problem, an additional component called an adjuvant is incorporated with the antigen in the vaccine. The function of this adjuvant is to “turn on” the immune system and help the immune system to mount a stronger response to the antigen.

There are few adjuvants approved for use in human vaccines. Most approved adjuvants induce protection by inducing antibodies that circulate in the subject’s blood, neutralising the microorganism. However, complex microorganisms, such as those causing influenza, tuberculosis or malaria, can escape these antibodies or antibodies alone cannot mediate protection. Therefore, protection against complex microorganisms requires an additional defence mechanism that is known as cell-mediated immunity and does not involve antibodies. It involves the activation of phagocytes, cytotoxic T lymphocytes and various cytokines in response to antigen. There are several types of types of effector CD4+ T helper cell responses that can be induced by an Antigen Presenting Cell (APC), these include responses called a Th1 and Th2 response. A Th1 response is effective against intracellular pathogens (viruses and bacteria that are inside host cells) and destroys infected cells and is a potent defence against cancer. CD8+ T cells also play an important role in cell mediated immunity against intracellular pathogens, including viruses and bacteria, and for tumour surveillance. Currently there is a lack of effective adjuvants able to promote these types of protective cellular immunity.

Polymeric nanoparticles, including poly(D, L-lactide-co-glycolide)-PLGA polymer particles, have been tested as adjuvants in many preclinical scenarios. However, despite the efforts of many research groups, there is no report of a PLGA nanoparticle that could promote the desired cell mediated immunity more efficiently than adjuvants in clinical use. Furthermore, currently reported PLGA particles require co-adjuvants or complex encapsulation, in order to elicit such an effect.

While PLGA particles have been effectively used as drug carriers, they have not been effectively used in the vaccine industry. Generally, the induction of cell mediated immunity (CMI) reported had been poor. This has led to alternative co-formulation strategies with toll like receptor (TLR) agonists and other co-adjuvants or complex antigen encapsulation or conjugation methods that complicate formulation stability, increase the cost and challenge their translation into clinically relevant preparations.

Several groups have investigated PLGA nanoparticles and microparticles and their use for modulating immune responses.

CA2753567 discloses particles with a hydrophobic segment (PGLA) and a hydrophilic segment (refractory polysaccharide, e.g. dextran). The preferred diameter of these particles is stated to be between 0.1 µm to 50µm.

Oyewumi et al, (Expert Rev Vaccines, 2010) is a review of nanoparticles as immune adjuvants correlating particles sizes and the resultant immune responses. However, the authors conclude that overall, with the conflicting data, they could not achieve an accurate prediction of particle size ranges that will dictate a Th1 or a mixed Th1/Th2 immune response outcome.

CA02731995 discloses a method of inducing a Th1 immune response using microparticles. The particles are sized such that at least 50% are less than 5um, preferably less than 3µm. It further states that the mean diameter of the microparticles is greater than or equal to 2.2µm.

WO2012054425 discloses a composition for therapy of tumours and the aim of this study was to produce a strong Th1 response. It discloses particles, preferably made from PLGA. The average diameter is about 100nm to 20µm, 200nm to 15µm, most preferably 500nm to about 10µm. The particle used also requires a co-adjuvant to elicit an effect.

O’Grady et al., (J Immunol, 2019) discloses polystyrene nanoparticles that are drivers of antigen-specific cellular immunity following vaccination.

Chenxi Li, et al (Applied materials and interfaces, 2018, 10, 2874-2889) discloses amphiphilic diblock copolymer poly(2-ethyl-2-oxazonline) poly (D, L-lactide) (PEOz-PLA) combined with carboxylterminated pluronice F127 to construct nanoparticles for the delivery of antigen ovalbumin. This system includes a co-adjuvant, namely TLR7, in order to elicit a response.

WO2017/151922 discloses vaccines comprising an antigen and a copolymer, in which the antigen is in the core of the polymer particle. An organic polymeric nanoparticle as a delivery agent is also disclosed by US2015/0342883.

Carmen Garcia Arevelo, et al.,(Molecular Pharmaceutics, 2013, 10, 586-597) discloses a genetically engineered elastin like block corecombinator (ELbcR) that allows the production of antigenic particle based constructs that assemble into nanovesicles. A Th2/IL-5 response was reported in mice.

Fazren Azmi, et al., (Bioorganic and Medicinal Chemistry) discloses conjugation of lipid moieties to peptides prompting a non-polymeric particle formation and use in vaccine constructs.

In addition to PLGA several other particulate systems, such as those described in Schiins et al (Immunol Rev. 2020 Jul;296(1):169-190), have been shown to have certain adjuvant activity, but most are very complex formulations that require multiple components in addition to antigen encapsulation, or covalently bound antigens, to work. Complex multicomponent particle products are impractical for mass production and represent a disadvantage for the vaccine industry. Another general problem with such systems is the lack of knowledge regarding their mechanisms of action or the immune pathways they require to be effective adjuvants.

The current invention serves to address the problems of the prior art by providing a biocompatible nanoparticle that can be used as an adjuvant to promote a cell mediated CD8 and/or Th1 response without the need for a co-adjuvant.

### SUMMARY OF THE INVENTION

The current invention provides a pure, endotoxin free, polymeric nanoparticle that successfully and surprisingly induces an effective Th1 and cytotoxic T lymphocyte (CTL) response, using a mixed antigen. The nanoparticle is capable of inducing the response without a co-adjuvant. This has not been previously reported or predicted in the prior art. The invention represents an advantage in terms of simplicity of formulation.

The current inventors have shown that the PLGA particles are potent inducers of CD8 T cells and antigen-specific secretion of the cytokine IFN-_{Y}. IFN- _{Y} is the hallmark of the Th1 response. The current inventors have further shown that this response cannot be driven by larger biodegradable PLGA or larger biocompatible polystyrene particles.

Therefore, the current invention offers a novel strategy to induce potent CD8 or Th1 responses via vaccination using purified antigens and biodegradable PLGA particles that has not been described before.

The current inventors have also shown that the ability of the 50nm nanoparticles of the invention to induce CD8 responses is dependent on a pyroptotic related pathway. This pathway can lead to programmed immunogenic cell death and/or hyperactivation of cells.

Prior to the current invention, limitations in the PLGA particle formulation processes restricted their production to sizes between 80nm and 250µm. New platforms including the microfluidic technology applied to manufacture the 50nm particles used in the current invention allows for fine control of particle size, particles with a low polydispersity index, avoiding the use of harmful solvents and allowing escalation for mass production of adjuvants.

An aspect of the invention provides a polymeric nanoparticle having a diameter of less than 80nm, for use in inducing a CD8 response and/or a Th1 response in a subject.

The polymeric nanoparticle for use is as described herein and referred to as “nanoparticle of the invention”.

The response is against an immunogenic species in a subject. Typically, the immunogenic species is an antigen.

Preferably, the use is inducing a CD8 response in a subject to the antigen. Typically, the nanoparticle induces, or affects, a pyroptosis-related pathway in a subject.

In an embodiment, a CD8 response and a Th1 response is induced.

Preferably, the polymeric nanoparticle has a diameter of from about 30nm to about 65nm, preferably from about 40nm to about 60nm.

Typically, in said use the polymeric nanoparticle is mixed with the immunogenic species.

Preferably, the immunogenic species is adsorbed to the surface of the nanoparticle and/or free in a composition or formulation comprising said nanoparticle.

Most preferably, the polymer nanoparticle for use is biocompatible.

Typically, the nanoparticle for use is endotoxin free.

Notably, the nanoparticle for use is a solid particle.

In an embodiment, the nanoparticle for use is provided in the form of a composition comprising the nanoparticle of the invention, or a preparation of nanoparticles of the invention. Typically, the composition also comprises the immunogenic species. The immunogenic species is mixed with the immunogenic species in the composition.

In an embodiment, the composition is a vaccine composition.

In an embodiment, the composition is an adjuvant.

Use of the nanoparticle of the invention as an adjuvant is also provided.

An aspect of the invention provides a polymeric nanoparticle (herein referred to as the “nanoparticle of the invention”) having a diameter of less than 80nm, preferably, from 40nm to 60nm, still preferred about 52nm to about 65nm, or 52nm to 60nm.

The polymer nanoparticle is biocompatible.

In an embodiment, the nanoparticle of the invention comprises a biocompatible polymer. Preferably, the nanoparticle is a polymeric particle. Preferably, the nanoparticle is a poly(D, L-lactide-co-glycolide) (PLGA) polymer nanoparticle. The nanoparticle is a polylactic acid (PLA) nanoparticle. Typically, the biocompatible polymer is selected from the group comprising PLGA, PLA, polyphosphazene and chitosan.

An aspect of the invention provides a preparation of biocompatible polymeric nanoparticles of the invention (“preparation of nanoparticles of the invention”).

In an embodiment, at least 90% of the nanoparticles in the preparation have a diameter of less than 80nm, preferably, from 40nm to 60nm.

In an embodiment, at least 90% of the nanoparticles in the preparation have a diameter of from about 52nm to 65nm, preferably 52nm to 60nm.

Preferably, the nanoparticles in the preparation are uniform or monodisperse.

As aspect of the invention provides a composition comprising the nanoparticle of the invention.

Typically, the composition comprises at least one immunogenic species.

In an embodiment, the composition is a vaccine.

In an embodiment, the composition is a pharmaceutical composition.

In an embodiment, the immunogenic species is an antigen.

Typically, the polymeric nanoparticle is mixed with the immunogenic species in the composition.

Preferably, the immunogenic species is adsorbed to the surface of the nanoparticle and/or free in a composition.

In an embodiment of the invention, at least 90% of the nanoparticles in the composition have a diameter of less than 80nm.

In an embodiment of the invention, at least 90% of the nanoparticles in the composition have a diameter from 40nm to 60m, or from about 52nm to about 65nm, preferably 52nm to 60nm.

In an embodiment, the composition comprises a plurality of nanoparticles.

In an embodiment, the composition further comprises a pharmaceutically acceptable carrier or salt.

An aspect of the invention provides an adjuvant comprising the nanoparticle of the invention.

An aspect of the invention provides the nanoparticle, preparation or composition of the invention and as described herein for use in vaccine therapy to prevent or treat a condition or disease in a subject. In an embodiment, the disease or condition is a bacterial infection or a viral infection. Preferably, the disease or condition is cancer.

The disease or condition may be a chronic infectious disease. The chronic infectious disease may be selected from, but not limited to, tuberculosis, viral infections, intracellular bacteria, and intracellular parasites.

An aspect of the invention provides a method of treating or preventing a disease or condition in a subject, comprising a step of administering to the patient a therapeutically or prophylactically effective amount of the nanoparticle, preparation, or composition of the invention.

A further aspect of the invention provides the nanoparticle, the preparation or composition of the invention and as described herein, for use in inducing a Th1 response and/or a CD8 response in a subject to an antigen.

A further aspect of the invention is a method of producing a Th1 and/or a CD8 response in a subject to an antigen, comprising administering a therapeutically effective amount of the nanoparticle, the preparation or composition of the invention to said subject.

A further aspect of the invention provides the nanoparticle, preparation or composition of the invention as a carrier to deliver an active agent to a subject. The active agent may be a drug, such as a drug for cancer treatment, preferably a chemotherapeutic drug.

### DEFINITIONS AND GENERAL PREFERENCES

Where used herein and unless specifically indicated otherwise, the following terms are intended to have the following meanings in addition to any broader (or narrower) meanings the terms might enjoy in the art:

All references cited in herein are incorporated herein by reference in their entireties.

Unless otherwise required by context, the use herein of the singular is to be read to include the plural and vice versa. The term “a” or “an” used in relation to an entity is to be read to refer to one or more of that entity. As such, the terms “a” (or “an”), “one or more,” and “at least one” are used interchangeably herein.

As used herein, the term “comprise,” or variations thereof such as “comprises” or “comprising,” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein the term “comprising” is inclusive or open-ended and does not exclude additional, unrecited integers or method/process steps.

The term “cell mediated immunity” when used herein, refers to the production of cytokines, chemokines and other such molecules produced by activated T-cells and/or other white blood cells, including those derived from CD4+ and CD8+ T-cells.

The term “nanoparticle” as used herein, refers to a particle of less than 1,000 nm in diameter. The nanoparticle may be solid or hollow. The nanoparticle may be porous.

The term “immune response” should be understood to mean induced humoral and/or cellular response in a subject.

The term “antigen” when used herein means a substance that, when introduced in the body, induces an immune response in the body. The term may be used interchangeably with the term “immunogen.”

In this specification, the term “vaccine” should be understood to mean a composition or formulation comprising the nanoparticle of the invention and at least one antigen. The preparation of vaccines is well described in the literature, for example US4599230 and US4601903, the complete contents of which are incorporated herein by reference.

The term “adjuvant” should be understood to mean an agent that enhances the recipient’s immune response to an antigen.

The term “therapeutically effective amount” should be taken to mean an amount which results in a clinically significant treatment, reduction or prevention of the disease or condition. The amount will vary from subject to subject, depending on the age and general condition of the individual, mode of administration and other factors. Thus, while it is not possible to specify an exact effective amount, those skilled in the art will be able to determine an appropriate “effective” amount in any individual case using routine experimentation and background general knowledge. A therapeutic result includes eradication or lessening of symptoms, reduced pain or discomfort, prolonged survival, improved mobility and other markers of clinical improvement. A therapeutic result need not be a complete cure.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans.

The term “polymeric material” when used herein is a material comprising one or more polymers.

“PLGA” is a copolymer commonly synthesized by means of ring-opening co-polymerization of two different monomers, the cyclic dimers (1,4-dioxane-2,5-diones) of glycolic acid and lactic acid. PLGA is usually identified in regard to the molar ratio of the monomers used, e.g. PLGA 75:25 identifies a copolymer whose composition is 75% lactic acid and 25% glycolic acid.

The term “carrier” refers to a diluent, adjuvant, excipient, or vehicle with which the therapeutic is administered. Typically, it is a pharmaceutically acceptable carrier. Such carriers can be sterile liquids, such as water and oils, including those of petroleum, animal, vegetable or synthetic origin, such as peanut oil, soybean oil, mineral oil, sesame oil and the like. Water is a preferred carrier when the formulation or composition is administered intravenously. Saline solutions and aqueous dextrose and glycerol solutions can also be employed as liquid carriers, particularly for injectable solutions. The carrier may be particularly for human therapy. Acceptable carriers for therapeutic use are well known in the pharmaceutical art, and are described, for example, in Remington’s Pharmaceutical Sciences, Mack Publishing Co. (A. R. Gennaro edit. 1985). Examples of suitable carriers include sucrose, lactose, starch, glucose, methyl cellulose, magnesium stearate, mannitol, sorbitol and surfactants, such as DOTAP and phosphatidylserine and the like. Examples of suitable diluents include ethanol, glycerol, water, PBS, Tris Buffer saline, and other physiological buffers. The choice of pharmaceutical carrier can be selected with regard to the intended route of administration and standard pharmaceutical practice. Preferably, any carrier included is present in trace amounts. The nature and amount of any carrier should not unacceptably alter the benefits of the antigens of this invention.

The term “excipient” refers to any essentially accessory substance that may be present in the finished formulation of the invention. Typically, it is a pharmaceutically acceptable excipient. For example, the term “excipient” includes but is not limited to vehicles, binders, disintegrants, fillers (diluents), lubricants, suspending/dispersing agents, coating agent stabilisers, dyes, emulsifying agents, emollients, preservatives, and/or surfactants. Suitable excipients include, but are not limited to, starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The excipient may be particularly for human therapy. Examples of such suitable excipients for the various different forms of pharmaceutical compositions described herein may be found in the “Handbook of Pharmaceutical Excipients”, 2^{nd} Edition, (1994), edited by A Wade and PJ Weller. The choice of pharmaceutical excipient can be selected with regard to the intended route of administration and standard pharmaceutical practice.

The term “surfactant” includes non-ionic surfactants, cationic surfactants, anionic surfactants, and zwitterionic surfactants, among others. Surfactants may be added, for example, to ensure that lyophilized nanoparticles can be resuspended without an unacceptable increase in size (e.g., without significant aggregation). The surfactant may be particularly for human therapy.

Preservatives, stabilizers, or dyes may be provided in the composition or formulation. Examples of preservatives include sodium benzoate, sorbic acid and esters of phydroxybenzoic acid. Antioxidants and suspending agents may be also used.

The term “pharmaceutically acceptable” means approved by a regulatory agency of the Federal or a state government or listed in the U.S. Pharmacopeia or other generally recognized pharmacopeia for use in animals, and more particularly in humans.

Formulation or compositions of the invention can be prepared with a carrier that will protect the compound against rapid release, such as a controlled release formulation, including implants, transdermal patches, and microencapsulated delivery systems. Methods for the preparation of such compositions are known. Pharmaceutically acceptable substances, which desirably can enhance the shelf life or effectiveness of the formulation or composition are provided.

In practicing the invention, the amount or dosage range of the antigen of the invention employed typically is one that effectively induces, promotes, or enhances a physiological response associated with the nanoparticle, formulation or composition of the invention.

When used herein the term “cancer” may be selected from the group comprising but not limited to, gastrointestinal cancer, head and neck cancer, cancer of the nervous system, kidney cancer, renal cell carcinoma, retinal cancer, melanoma, stomach cancer, liver cancer, genital-urinary cancer, colorectal cancer, and bladder cancer, multiple myeloma, glioblastoma, lymphoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing’s tumour, leiomyosarcoma, rhabdomyosarcoma, colon carcinoma, pancreatic cancer, breast cancer; ER-positive breast cancer; ovarian cancer; squamous cell carcinoma; basal cell carcinoma, adenocarcinoma, sweat gland carcinoma, sebaceous gland carcinoma; papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms’ tumour, cervical cancer, uterine cancer, testicular tumour, lung carcinoma, small cell lung carcinoma, bladder carcinoma, epithelial carcinoma, glioma; astrocytoma, medulloblastoma, craniopharyngioma, ependymoma, pinealoma, hemangioblastoma, acoustic neuroma, oligodendroglioma, meningioma, retinoblastoma, and leukemias, epithelial cancer, solid tumour and haematological malignancies, metastatic cancer. Typically, treatment of the cancer entails reducing one or more of reducing survival, proliferation and migration of, or invasion by cancer cells.

In this specification, the term “composition” should be understood to mean something made by the hand of man, and not including naturally occurring compositions. The composition may be for human or animal use.

The term “mammal” should be understood to mean a higher mammal, especially a human. However, the term also includes non-mammalian animals such as fish. The human may be an infant, toddler, child, adolescent, adult, or elderly human. The mammal may be an animal.

The terms “treat,” “treatment,” and “treating,” as used herein, refer to an approach for obtaining beneficial or desired results, for example, clinical results.

The term “prevention,” as used herein, is used interchangeably with “prophylaxis” and can mean complete prevention of an infection or disease, or prevention of the development of symptoms of that infection or disease; a delay in the onset of an infection or disease or its symptoms; or a decrease in the severity of a subsequently developed infection or disease or its symptoms.

As used herein, the term “vaccine” refers to an immunogenic composition, such as the formulation of the invention, which is used to induce an immune response that provides protective immunity (e.g., immunity that protects a subject against infection with the pathogen and/or reduces the severity of the disease or condition caused by infection with the pathogen, or that prevents the survival, spread or growth of cancer cells).

As used herein, an “immunogenic composition” is a composition that comprises the nanoparticle of the invention and an antigen where administration of the composition to a subject results in the development in the subject of a cellular immune response to the antigen. The term may be used interchangeably with “vaccine formulation” in this context.

As used herein, “polydispersity index” (PDI) is a measure of the heterogeneity of a sample based on size. Polydispersity can occur due to size distribution in a same. Methods to measure polydispersity index are known in the art. One means to calculate the PDI is from the cumulative analyses carried in a machine called zetasizer that uses dynamic light scattering. PDI can adopt values between 0 and 1. The lower the polydispersity index the more homogenous nanoparticles. The closer the value is to 1 the bigger the variation in particle size. In this context, the nanoparticles of the invention, or the nanoparticles in the preparation of the invention typically have a PDI 0.26+/-0.10 or lower.

As used herein “CD8 T cell response” is the induction of cytotoxic T cells expressing the CD8 coreceptor and capable of killing cells, proliferating, and producing cytokines on recognition of cells expressing their target antigen

As used herein “Th1 response” is the induction of Th1 cells, a lineage of CD4+ T cells that promote cell mediated immunity to intracellular pathogens and cancer. Th1 cells produce specific cytokines, particularly interferon-gamma which are important for their function.

### BRIEF DESCRIPTION OF THE FIGURES

The invention will be more clearly understood from the following description of an embodiment thereof, given by way of example only, with reference to the accompanying drawings, in which:

FIG. 1: 50nm PLGA nanoparticles promote CD8⁺ T cell responses and antigen-specific IFN-_{Y} when administered with a protein antigen. C5/BL/6 mice were immunized on days 0 and 14 with positively charged (PLGA-DOTAP) or negatively charged PLGA-PhS) particles and ovalbumin (OVA) via the intramuscular route. On day 21 the splenocytes were analysed by flow cytometry to quantify the percentage (A) and number (B) of CD8⁺ T cells specific to the H-2 Kb OVA epitope SIINFEKL using tetramers. In all cases 50nm polystyrene (PS) particles were used as positive control. Results are shown as mean±SEM and symbols represent individual animals. (C) IFN-_{Y} secretion was analyzed by ELISA after 72h restimulation with the antigen.

Asterisks denote statistical differences calculated as per multiple comparisons ANOVA and Dunnett’s multiple comparison test to determine differences compared to OVA alone.

FIG. 2: Bigger PLGA particles fail to induce CD8⁺ T cell responses and IFN-_{Y} when administered with a protein antigen. C57BL/6 mice were immunized on days 0 and 14 with particles and ovalbumin (OVA) via the intramuscular route. On day 21 the splenocytes were analysed by flow cytometry to quantify the percentage (A) and number (B) of CD8⁺ T cells specific to the H-2 Kb OVA epitope SIINFEKL using tetramers. Results are shown as mean±SEM and symbols represent individual animals. (C) IFN-γ expression was analysed by ELISA after 72h restimulation with the antigen. Polystyrene nanoparticles were used as a positive control (+Ctrl); anti-CD3 stimulation was used to confirm IFN-γ production in all groups (right panel).

Asterisks denote statistical differences calculated as per multiple comparisons ANOVA and Dunnett’s multiple comparison test to determine differences compared to OVA alone.

FIG. 3: 50nm nanoparticles induce antigen-specific CD8 T cell responses through an immunogenic cell death pathway. Mice were immunized via the intramuscular (i.m.) on day 0 and 14 with OVA alone or with particles (PS). Mice were treated with 250µg of necrosulfonamide (NSF) to inhibit pyroptosis by the intraperitoneal (i.p.) or i.m. route during priming and booster. On day 21 flow cytometry was used to determine the frequency of splenic CD8⁺ T cells specific to the H-2 Kb OVA epitope SIINFEKL using tetramers Results are shown as mean±SEM and symbols represent individual animals. Asterisks denote statistical differences calculated as per multiple comparisons ANOVA and Dunnett’s multiple comparison test to determine differences compared to OVA + 50nm PS.

FIG. 4: Caspase 11 is required for nanoparticle-induced antigen-specific CD8 responses. Caspase-11 knockout (Casp11 KO) or Wild-type (WT) mice were vaccinated intramuscularly on days 7 and 14 with 50nm particles plus OVA; OVA alone or PBS as vehicle control. On day 21 antigen specific responses were quantified in the spleen using H-2Kb/OVA (SIINFEKL) MHC Tetramers. (A) percentage of OVA-specific CD8+ over total CD8+ T cells; (B) Total number of OVA-specific CD8+ T cells in the spleen. Statistical differences as per one-way ANOVA and Bonferroni post-hoc test. Asterisks indicate significant differences ** P<0.01, * P<0.05.”

FIG. 5: Characteristics of nanoparticles formulated using PLGA 50:50, PLGA:DDA (dioctadecyl ammonium bromide) or PLGA PhS (Phosphatidylserine) 1:1 w/w. After formulation, nanoparticles were characterised using a Zetasizer Nano Z system to measure zeta potential and electrophoretic mobility in aqueous dispersions using Laser Doppler Micro-Electrophoresis. Size (nm), polydispersion index (PDI) and zeta potential (ZP) are shown in the table. Results are presented as average and standard deviation (SD) and are representative for at least two separate batches of nanoparticles.

FIG. 6: Illustrates a potent antigen specific anti-tumour immunity driven by 50nm PS nanoparticles. A) Tumour growth rate at challenge site of mice bearing tumours, displayed as mean tumour volume ± SEM. A 2 way ANOVA with Tukeys multiple comparisons test was used to determine statistical significance between PBS and 50nm vaccinated mice, where *p<0.05, **p<0.01, and ***p<0.001. (n=7 mice per group). B) Kaplan-Meier survival analysis of challenged mice. A Mantel-Cox test was used to determine statistical significance between PBS and 50nm vaccinated mice where *p<0.05, **p<0.01, and ***p<0.001.

FIG. 7: Injection of polymeric particles enhances antigen-specific antibody responses independently of size. C57BL/6 mice were immunized with endotoxin-free ovalbumin (OVA) or OVA and particles of different sizes on days 0 and 14 according to previously optimized does for (A) intramuscular (10µg OVA+1 mg particles or alum) or (B) intraperitoneal (1µg OVA, 4mg particles or alum). For subcutaneous immunization either OVA (C) or Staphylococcal Clumping factor A -ClfA (D, E) were used as antigens (50µg OVA or 1µg ClfA, 4mg particles). PBS was used as vehicle control and Alhydrogel® (alum) was used as a gold standard control for i.m. and i.p.. On day 21, blood samples were collected and antigen-specific IgG titers were measured in serum by ELISA. Neutralizing antibodies were determined by testing the ability of serum from vaccinated mice to inhibit the adherence of S. aureus to fibrinogen at 37° C. by measuring Abs570nm in fixed bacteria previously stained with crystal violet. Adherence is expressed as percent inhibition related to the maximum (100%) calculated as bacterial adherence in the absence of serum. Results are shown as mean±SEM and each symbol represents an individual animal. Asterisks represent statistical differences (p>0.0001) calculated as per multiple comparison ANOVA and Dunnette’s test to determine differences compared to antigen alone. Specific antibodies were not detected in mice injected with PBS alone. Representative of 2 experiments via i.p. and s.c. and 1 experiment via i.m. route.

FIG. 8: Particle size influences antibody class-switching but does not affect antibody neutralizing activity. Mice were immunised with particles and OVA via intramuscular route (A-C) or subcutaneously with particles and ClfA (D-G) on days 7 and 14. Serum was collected on day 21 for analysis of antigen specific IgG isotypes by ELISA. Results are shown as mean±SEM with symbols representing individual animals. Asterisks denote statistical differences calculated as per multiple comparison ANOVA and Dunette’s test to determine differences compared to antigen alone. Representative of 2 experiments.

FIG. 9: 50nm particles promote antigen-specific IFN-γ secretion required for IgG2c class-switching. Mice were immunized on day 0 and 14 by intramuscular route with particles and OVA. On day 21 spleens and serum were collected. (A) Splenocytes were restimulated with OVA, anti-CD3 as a positive control (inner panel) or left unstimulated. IFN-γ production was determined by ELISA. Bars represent mean±SEM for n=4/group. (B) OVA-specific total IgG titers and isotypes were determined by ELISA in serum. Results are shown as mean±SEM and symbols represent individual animals. In all panels asterisks denote statistical differences calculated as per multiple comparison ANOVA and Dunnette’s multiple comparison test to determine differences compared to OVA alone.

FIG. 10: CD8⁺ T cell responses are exclusively induced by 50nm particles independently of administration route or polymer. Mice were immunized on day 0 and 14 by i.m. (A) or i.p (B) routes with OVA alone or combined with alum or particles. On day 21 the splenocytes were analyzed by flow cytometry to quantify the number of CD8+ T cells specific to the H-2 Kb OVA epitope SIINFEKL using tetramers. Asterisks denote statistical differences calculated as per multiple comparison ANOVA and Dunnette’s multiple comparison test to determine differences compared to OVA alone. Representative of 1 experiment via i.m. and 2 experiments i.p. The efficacy of 50nm polystyrene (PS) and 50nm poly(lactic-co-glycolic acid) (PLGA) was compared after i.m. injection following the same vaccination scheme as before; (C) On day 21 the splenocytes were analyzed by flow cytometry to quantify the number of CD8⁺ T cells specific to the H-2 Kb OVA epitope SIINFEKL using tetramers (shows mean±SEM) and (D) representative dot plots. (E) shows mean±SEM SIINFEKL-specific CD8+, CD44+ T cells after vaccination with 50nm PS, 100nm PLGA or 500nm PLGA with respective dot plots (F). Antigen-specific IFN-_{Y} response measured after ex vivo stimulation of splenocytes by ELISA to compare 50nm PS vs 50nm PLGA (G) and 50nm PS vs 100nm and 500nm PLGA (H). Asterisks denote statistical differences calculated as per multiple comparison ANOVA and Dunnette’s multiple comparison test to determine differences compared to OVA alone. Representative of 1 experiment via i.m. and 2 experiments i.p..

FIG. 11: 50nm particles induce antigen-specific CD4⁺ T cells and long term CD8⁺ T cell memory. (A) WT mice were immunized i.p. on days 7 and 14 using the I-Ab restricted influenza nucleoprotein peptide NP₃₁₁₋₃₂₅ alone or in combination with PS particles of different sizes or alum as a positive control. Antigen-specific CD4⁺ responses were assessed by FACS using class II (C-II) tetramers (Tmer) on day 21 in spleens. Vaccination with 50nm PS particles effectively increased the number of antigen-specific CD4⁺ T cell responses. (B) 50nm PS also enhanced NP₃₁₁₋₃₂₅ CD4⁺ T cell responses after i.m. vaccination 7 days after booster using a fusion antigen generated by conjugation of NP₃₁₁₋₃₂₅ to OVA (OVA-NP₃₁₁). (C) The number and percentage of H-2 Kb OVA epitope SIINFEKL CD8⁺ T cells was also upregulated 7 days after i.m. prime/boost vaccination with 50nm PS and the fusion NP₃₁₁-OVA antigen. (D) Upper left graph shows Ag-specific CD8⁺ memory T cells were detected in spleens 100 days after prime/boost i.m. vaccination with PS+ NP₃₁₁-OVA. Dot plots show C-I Tmer⁺ CD8⁺ T cells over total CD8⁺ lymphocytes. Blue histograms show surface marker expression for CD69, CD103 and CD62L in C-I Tmer⁺ memory CD8+ T cells in spleens; grey histograms show unstained control for each marker. Lower left graph shows IFN-_{Y} production measured by ELISA in splenocytes harvested on day 100 after vaccination and restimulated with the fusion antigen for 72h. Bars represent mean±SEM. Asterisks represent significant differences as per one-way ANOVA and multiple comparisons post-hoc Dunnett’s test, *P≤0.05, *P≤0.01. (A) Representative of 1 experiment with n=5/group, (B) representative of 2 experiments with n=3/group; (C) representative of 2 experiments, n=3/group; (D) representative of 1 experiment n=3/group.

FIG. 12: 50nm PS nanoparticles drive potent antigen specific anti-tumor immunity. Mice were vaccinated on days 0 and 14 with PBS, OVA (10µg) alone, or 50nm PS particles (1mg) plus OVA (10µg), followed by s.c challenge of 3.5x10⁵ B16-OVA tumor cells on day 28 as in (A). Tumors were measured daily. Spider plots (B) show early development of tumors measured as tumor volume in mm³. Each line represents an individual mouse. The percentage of mice bearing tumors and Kaplan-Meier survival graphs during early stages of the challenge are depicted in (C). Extended analysis of percentage of mice bearing tumors and survival analysis are presented in (D). For analysis of mice bearing tumors n=19 in PBS and OVA groups and 17 in 50nm+PS 50nm+OVA group. For survival n=14 was used for PBS and OVA and 12 for 50nm+OVA. Statistical significance for survival analyses was determined using Mantel-Cox test where *p<0.05, **p<0.01, and ***p<0.001. CP indicates number of animals that were completely protected from tumor challenge over total in each group

FIG. 13: IL-1 and IL-18 signaling regulate nanoparticle-induced IFN-_{Y} and CD8⁺ T cell responses respectively. WT or mice deficient in IL1-R1 or IL-18 were immunized as before via i.m. route with OVA alone or in combination with 50nm particles. (A and E) Antigen-specific humoral responses were measured by ELISA in serum obtained on day 21. (B,C and F,G) Graphs show the percentage and number of OVA H-2Kb CD8⁺ T cells in spleens of vaccinated mice, 7 days after booster. (D and H) Analysis of IFN-γ secretion in supernatants of splenocytes harvested 7 days after vaccination and stimulated with OVA for 72h ex vivo analyzed by ELISA. Asterisks denote statistical differences using an unpaired t-tests with Welch correction for comparison of OVA vs 50nm+OVA within each strain for ELISAs or calculated as per ANOVA and Dunnette’s multiple comparison test to determine differences between WT 50nm+OVA vs knockout mice that received the same treatment. Quantification of tetramer⁺ cells is shown as Tmer⁺, CD8⁺, CD44⁺ over total live CD8⁺. Asterisks denote significant differences to OVA alone, ns: not significant as per one-way ANOVA and Dunnette’s post-hoc test. Quantification of IFN-γ is shown as mean±SEM; significant differences are shown for 50nm+OVA vs OVA alone. *p<0.05, **p<0.01, and ***p<0.001 FIG. 14: Caspase 11 is required for antigen-specific CD8 responses. Caspase-11 knockout (Casp11 KO) or Wild-type (WT) mice were vaccinated intramuscularly with 1mg of 50nm particles plus 10mg of OVA, or particles, OVA and 0.25mg of caspase 1 inhibitor Y-VAD via intramuscular route on days 7 and 14. On day 21 antigen specific responses were quantified in the spleen using H-2Kb/OVA (SIINFEKL) MHC Tetramers. PBS or OVA alone were used as vehicle or antigen alone controls. (A) pecentage of OVA-specific CD8+ over total CD8+ T cells; (B) Total number of OVA-specific CD8+ T cells in the spleen. Statistical differences as per one-way ANOVA and Bonferroni post-hoc test. Asterisks indicate significant differences ** P<0.01, * P<0.05.”

FIG. 15: Polymeric nanoparticles induce antibody responses independently from caspase-1, caspase-11 or gasdermin D. C57BL/6 mice were immunized with endotoxin-free ovalbumin (OVA) or OVA and 50nm polystyrene particles on days 0 and 14 via intramuscular route (10µg OVA+1mg particles). PBS was used as vehicle control. On day 21, blood samples were collected and antigen-specific IgG titers were measured in serum by ELISA on day 28. (A) To assess the role of caspase-1 a group of mice received the caspase-1 inhibitor Ac-YVAD-cmk together with the vaccine at the time of prime and boost. (B) To investigate the role of caspase-11, a group of caspase-11 deficient mice was vaccinated alongside wild-types with the same formulation. (C) The role of gasdermin D in humoral responses was assessed by evaluating antibodies in mice injected with the vaccine that received the Gasdermin-D inhibitor necrosulfonamide (NSF) either intramuscularly with the formulation or intraperitoneally at the time of vaccination during prime and boost.

Results are shown as mean±SEM and each symbol represents an individual animal. Asterisks represent statistical differences (p>0.0001) calculated as per multiple comparison ANOVA and Dunnette’s test to determine differences compared to antigen alone. Antibodies in the PBS group were taken as the baseline to define the detection limit.

FIG. 16: 50nm particles induce cell death in myotubes. C2C12 murine myoblasts were differentiated into myotubes and stimulated overnight with the indicated concentrations of polymeric particles of 50nm or 1µm. Cell death was measured as per LDH release assay and expressed as percentage of cytotoxicity. Nigericin and LPS were used as positive control for cell death.

Other advantages and features of the present invention will become apparent from the following detailed description of various non-limiting embodiments of the invention. All references cited in herein, including the description, are incorporated herein by reference in their entireties.

### DETAILED DESCRIPTION OF THE INVENTION

One type of cell mediated immunity relies on generation of cells that are called CD8 T cells, which have cytotoxic activity, and Th1 cells, which secrete IFN-_{Y}. A Th1 response is necessary in certain acute and chronic diseases. However, many widely used adjuvants, including alum, bias the immune response towards a Th2 response and mainly promote antibody responses.

Currently, there is a lack of effective adjuvants that can promote a Th1 cell mediated response. Likewise, current clinical adjuvants fail at inducing functional CD8 T cell responses as these responses typically require a higher activation threshold with simultaneous activation of CD4 T cells and dendritic cells.

CD8 T cells are needed for protection against intracellular pathogens including viruses, bacteria, mycobacteria and parasites but can also contribute to defences against cancer. CD8 T cells have the ability of directly killing infected or malignant cells and can also provide cytokines that activate the immune system. Pyroptosis is a form of programmed cell death, which is associated with cell membrane pore formation, cytoplasmic swelling, membrane rupture and the release of cytosolic contents, such as IL-1β and IL-18 into the extracellular environment, amplifying the local or systemic inflammatory effects. Pyroptosis is thought to be mediated by gasdermins. The activation of the pyroptosis molecular pathways involves activation of caspase-11, which subsequently activates the pore forming protein Gasdermin D (GSDMD). The activation of the pyroptosis pathway components does not always lead to cell death but has been shown to promote a hyperactivation state in cells via generation of reactive oxygen species. Both cell death or hyperactivation of the cells induce the release of endogenous immunogenic molecules that can activate the immune system and promote potent immune responses. This pathway has been shown to promote cellular immunity during infections but until now, no particulate adjuvants have been shown to promote it. The current inventors have surprisingly found that the ability of the particles to induce a pathway involved in a type of cell death called pyroptosis is key to their ability to promote CD8 responses.

The current invention solves the problems of the prior art by providing an adjuvant that can surprisingly promote a CD8 T cell and/or Th1 cell mediated immune response in a subject to an immunogenic species.

In an embodiment, the CD8 T cell response is one mediated by caspase 11 or gasdermin D or by both caspase 11 and gasdermin D. In one embodiment, the CD8 T cell response is one mediated by IL-1 and/or IL-18.

In one embodiment, the CD8 T cell response is one mediated by caspase 11, gasdermin D, IL-1 and IL-18.

In an embodiment, the Th1 response is one mediated by caspase 11 or gasdermin D or by both caspase 11 and gasdermin D. In one embodiment, the Th1 response is one mediated by IL-1 and/or IL-18.

In one embodiment, the Th1 response is one mediated by caspase 11, gasdermin D, IL-1 and IL-18.

The adjuvant can elicit this effect without the need for a co-adjuvant. This contrasts with the polymeric particles of the prior art, which require a co-adjuvant in use. This simplifies formulation and limits unwanted inflammatory responses, or toxicities, which have been linked to co-adjuvants, which may be efficient but also significantly reactogenic, such as agonists of Toll like receptors.

The current inventors have shown the precise size of nanoparticle that is able to induce a Th1 and CD8 protective response in a subject. This has not been reported previously.

The adjuvant is a polymeric nanoparticle (herein referred to as “the nanoparticle of the invention”).

The invention provides a biocompatible polymeric nanoparticle (i.e., the nanoparticle of the invention), for use in inducing a CD8 response and/or a Th1 response in a subject. The response is one against an immunogenic species. In an embodiment, both a CD8 response and Th1 response is elicited. This is particularly beneficial for cancer and viral vaccine use.

The immunogenic species is as described here.

The nanoparticle of the invention comprises a biocompatible polymer. Any suitable polymer may be used. The biocompatible polymer may be selected from the group comprising, but not limited to, PLGA, PLA, polyphosphazene and chitosan. Preferably, the nanoparticle is a poly(D, L-lactide-co-glycolide) (PLGA) polymer nanoparticle.

The nanoparticle of the invention is small, preferably having a diameter (i.e. particle size) of from about 30nm to about 80nm in size. Preferably, the particle has a diameter of from about 40nm to 60nm, or 50nm to 60nm in size, typically, 35nm, 40nm, 45nm, 50nm, 51nm, 52nm, 53nm, 54nm, 55nm, 56nm, 57nm, 58nm, 59nm, 60nm, 61nm, 62nm, 63nm, 64nm, 65nm, 70nm or 75nm, in size. It may be less than or equal to these values.

The nanoparticle of the invention may be biodegradable. It may be nondegradable.

The nanoparticle of the invention may have a PDI of 0.36 or less, preferably 0.26 or less. It may be between 1.6 and 3.6. An advantage of having a low polydispersity means that most of the particles are of the ideal size to enhance CD8 and/or Th1 responses. This also has potential to reduce the adjuvant dose needed as the particles are all in the range to drive this beneficial response.

The nanoparticle of the invention may have a positive, neutral, or negative charge. Typically, the effect of the nanoparticle is independent of its charge.

The nanoparticle of the invention is uniform in size and shape. Typically, the nanoparticle is substantially spherical.

The nanoparticle of the invention may be a plurality of nanoparticles.

The nanoparticle of the invention may further comprise a co-adjuvant. Co-adjuvants are known in the art and all suitable co-adjuvants are encompassed herein.

The nanoparticle of the current invention is endotoxin free. PLGA manufacturing methods reported in the prior art often introduce contaminants, such as endotoxin, which acts as a co-adjuvant. Being endotoxin free, also makes the particle of the current invention suitable for human use. Notably, the nanoparticle is a solid or hollow particle.

The nanoparticle used in the method of the invention or use of the invention may be a preparation of nanoparticles of the invention, or a composition comprising the nanoparticle of the invention. Both are as described herein.

In an embodiment, the invention provides a preparation of nanoparticles of the invention. Typically, the nanoparticles in the preparation have an average size, or size distribution in which the average value is, from about 30nm to 80nm, preferably from about 40nm to about 60nm, or 52nm and 65nm, typically from about 52 to 60nm. Preferably, at least 80% of the nanoparticles in the preparation have a diameter of from about 30nm to about 80nm in size, preferably from 40nm to 60nm. Preferably, at least 85%, 86%, 87%, 88%, 89%, 90%, 91%, 92%, 93%, 94%, 95%, 96%, 97%, 98%, 99% of the nanoparticles have a diameter of from about 30nm to about 80nm in size, preferably from 40nm to 60nm. Preferably, the particle has a diameter of from about 50 nm to 60nm in size, typically, 35nm, 40nm, 45nm, 50nm, 51nm, 52nm, 53nm, 54nm, 55nm, 56nm, 57nm, 58nm, 59nm, 60nm, 61nm, 62nm, 63nm, 64nm, 65nm, 70nm or 75nm, in size. It may be less than or equal to these values.

The invention provides a composition comprising the nanoparticle of the invention. The composition may further comprise at least one immunogenic species.

Typically, the immunogenic species is an antigen. Examples of antigens include polypeptide-containing antigens, polysaccharide-containing antigens, and polynucleotide-containing antigens, among others. Antigens can be derived, for example, from tumour cells and from pathogenic organisms such as viruses, bacteria, fungi and parasites, among other sources. It will be appreciated that the antigen may be any suitable antigen required by the circumstances.

The antigen may be any suitable antigen. Examples include but are not limited to tuberculosis antigens, such as H56, pneumococcal antigens, such as pneumolysin, PspA, conjugated pneumococcal polysaccharides, viral antigens, including flu antigens, such as influenza nucleoprotein, neuraminidase, hemagglutinin, or human papilloma virus proteins and peptides, tumour-derived antigens, such as Carcinoembryonic antigen (CEA), MelanA/Mart-1, MUC-1, NY-ESO-1, surviving, HER2/neu, GM2, among others or combinations thereof.

It will be understood that the composition may comprise a plurality of nanoparticles. Typically, the nanoparticles in the preparation have an average size, or size distribution in which the average value is, from about 30nm to 80nm, preferably from 40nm to 60nm, or about 52nm and 65nm, typically from about 52 to 60nm. Preferably, at least 80% of the nanoparticles in the formulation have a diameter of from about 30nm to about 80nm in size. Preferably, at least 85%, 86%, 87%, 88%, 89%, 90%, 91%, 92%, 93%, 94%, 95%, 96%, 97%, 98%, 99% of the nanoparticles have a diameter of from about 30nm to about 80nm in size, preferably from 40nm to 60nm. Preferably, the particle has a diameter of from about 50 nm to 60nm in size, typically, 35nm, 40nm, 45nm, 50nm, 51nm, 52nm, 53nm, 54nm, 55nm, 56nm, 57nm, 58nm, 59nm, 60nm, 61nm, 62nm, 63nm, 64nm, 65nm, 70nm or 75nm, in size. It may be less than or equal to these values.

The nanoparticle and the immunogenic species, which may be at least one antigen, may be combined in the composition by any suitable means known in the art. The particle may be mixed with the immunogenic species for example. In such an example, the nanoparticle and the antigen are not irreversible bound together and/or are non-encapsulated. In contrast to the prior art, in this embodiment there is no need for the immunogenic species to be encapsulated or irreversibly bound to the nanoparticle. In this embodiment the antigen may be adsorbed to the nanoparticle and/or mixed with it in the composition, i.e. the antigen may be free in the composition.

In an embodiment, the immunogenic species may be entrapped or encapsulated within the nanoparticle.

The immunogenic species may be attached to the nanoparticle.

The invention provides a composition comprising the preparation of the invention and at least one antigen.

The composition of the invention may be a vaccine. The vaccine of the current invention may be prophylactic. The vaccine of the current invention may be therapeutic. Notably, the composition of the current invention is suitable for anticancer and antiviral vaccines or vaccines against intracellular bacteria and parasites.

The composition may further comprise a pharmaceutically acceptable carrier or salt.

The composition of the invention may be a pharmaceutical composition.

In an embodiment, the composition may further comprise an adjuvant or co-adjuvant(s). Such adjuvants are known in the art and all suitable adjuvants are encompassed herein.

The composition may further comprise an active agent. The active agent may be any known active agent, such as a drug. The active agent may be a drug for cancer treatment, such as a chemotherapeutic.

The invention also provides a formulation comprising the nanoparticle of the invention. All features disclosed herein in relation to the composition of the invention equally apply to the formulation.

A composition comprising the nanoparticles of the invention or the preparation of the invention is provided. The composition may be an immunogenic composition or a pharmaceutical composition. In this particular embodiment of the invention, in use, the composition may be injected into a tumour and act with the endogenously released cancer antigens to elicit the Th1 and CD8 T cell response, rather than mixing the nanoparticle with the antigen before administration.

An adjuvant comprising the nanoparticle of the invention is provided. The nanoparticle may comprise at least one antigen.

A method of vaccinating a subject is also provided. The method involves administering the composition of the invention or the nanoparticle of the invention to said subject. The subject may be a human or animal. Delivery can be performed by any known method or as described herein.

A method of delivering an immunogenic species or antigen to a subject to induce an immune response to the species or antigen is also provided. The method comprises administration of the nanoparticle, preparation, or composition of the invention to a subject.

A method of delivering the nanoparticle, preparation, or composition of the invention to a subject is also provided. This may be for therapeutic, prophylactic or diagnostic purposes. The subject may be a human or animal. Delivery can be performed by any known method or as described herein.

The invention also provides a method of treating or preventing a disease or condition in a subject, comprising a step of administering to the subject a therapeutically effective amount of the nanoparticle, preparation or composition of the invention. The subject may be a human or animal. The diseases and conditions are as disclosed herein.

The invention also provides a method of inducing a Th1 response and/or CD8 T cell response in a subject. The method comprising administering to the subject a therapeutically effective amount of the nanoparticle, preparation or composition of the invention.

The invention also provides the nanoparticle, preparation or composition of the invention for use as an adjuvant in the treatment or prevention of a disease or condition in a subject.

The invention also provides the nanoparticle, preparation or composition of the invention for use in the treatment or prevention of a disease or condition in a subject. The subject may be a human or animal. The disease or condition may be a viral infection or a bacterial infection. The disease or condition may be any target cancer.

These diseases include but are not limited to viral infections such as SARS-CoV-2, influenza, hepatitis, herpes zoster, dengue, zika; or infections caused by intracellular bacteria eg. Listeria monocytogenes, Brucella abortus, or bacteria that require effective activation of macrophages for intracellular killing such as Group B streptococci, Staphylococcus aureus, Streptococcus pneumoniae, among others; mycobacteria including but not limited to Mycobacterium tuberculosis, intracellular parasites or parasites with intracellular developmental stages such as Toxoplasma gondii, Leishmania spp and diseases such as cancer. A Th1 response is necessary for treatment or prevention of these diseases.

Methods of introduction or administration include but are not limited to intradermal, intramuscular, intraperitoneal, intravenous, mucosal, subcutaneous, intranasal, e.g. aerosol, epidural, intracerebral, sublingual, intratumoral and oral routes. Typically, the nanoparticles or formulation of the invention are formulated for administration by injection. Naturally, for oral or sublingual, no injection is necessary. For intradermal administration, patches with microneedles may be used. The formulation or composition may be administered by any convenient route, for example by infusion or bolus injection, by absorption through epithelial or mucocutaneous linings (e.g., oral mucosa, rectal and intestinal mucosa, etc). Administration can be systemic or local. In addition, it may be desirable to introduce the formulation or composition of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent. Preferably, the method of administration is via intramuscular administration.

Methods to measure particle size are known in the art. The particle size of the polymer nanoparticles may be determined by dynamic light scattering (DLS) using a Malvern nano ZS. The method may be as described herein in the accompanying examples.

The nanoparticles of the invention may be prepared using any known suitable method in the art, such as microfluidic techniques. The method may be as described in the accompanying examples.

In use, the composition of the invention is used to vaccinate a subject. Following vaccination in a subject, the nanoparticle of the invention promotes the generation of CD8 T cells and/or Th1 cells in said subject. In particular, the small size of the particle allows this technical effect.

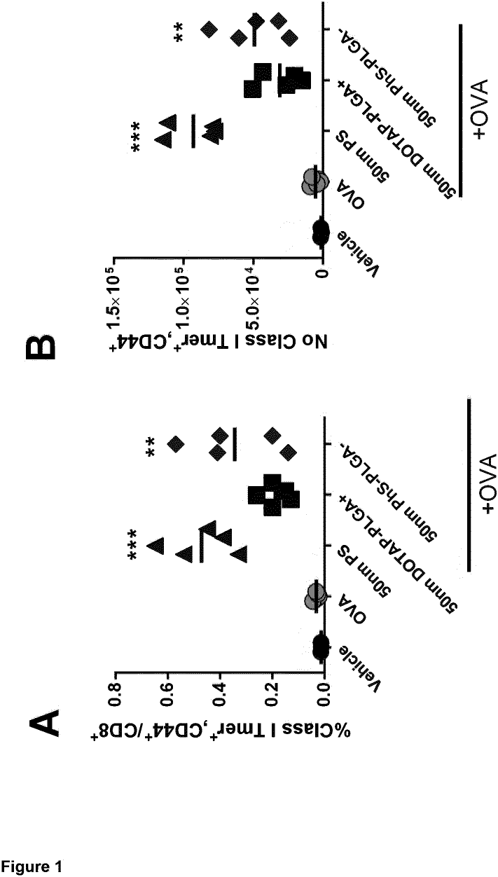

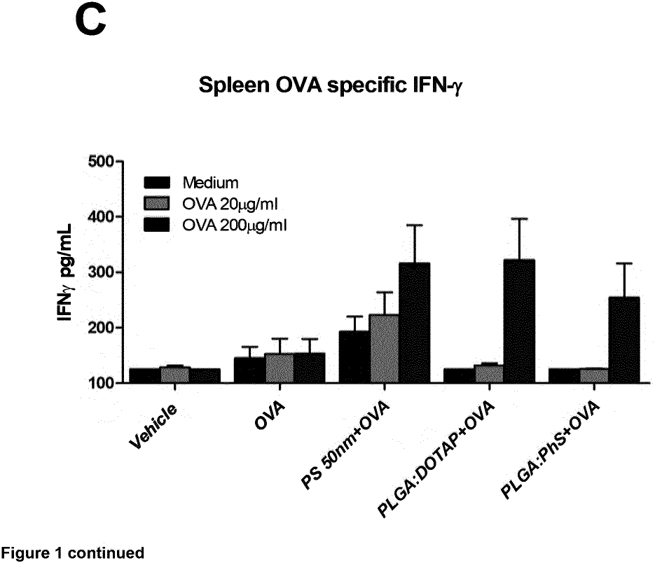

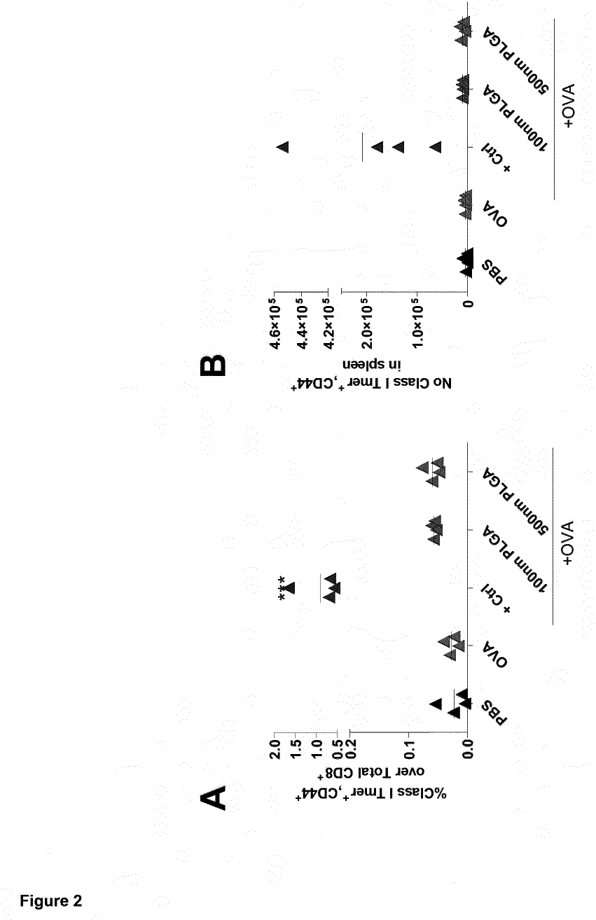

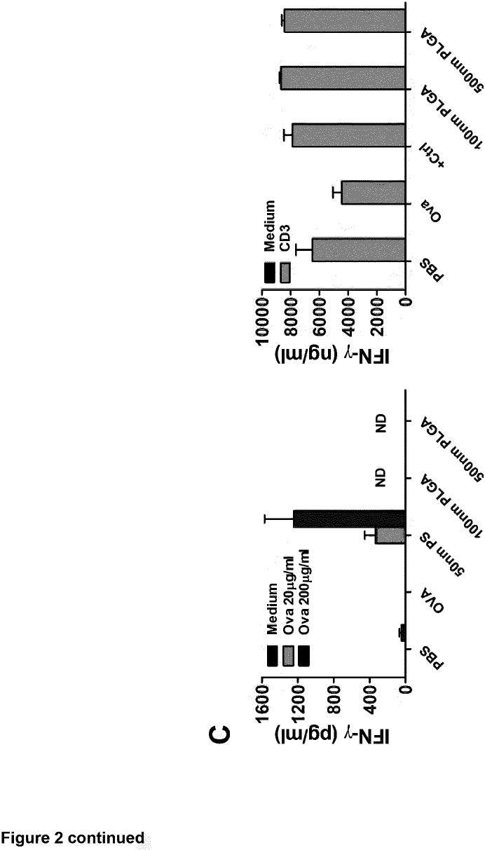

The current inventors have shown that the PLGA particles of the current invention are potent inducers of CD8 T cells and antigen-specific secretion of the cytokine IFN-_{Y}. IFN- _{Y} is the hallmark of the Th1 response. These results are illustrated in FIGS. 1A, B and C. The current inventors have further shown that this response cannot be driven by larger PLGA particles, as illustrated in FIGS. 2A and 2B. The current invention offers a novel strategy to induce potent CD8 and Th1 responses via vaccination using purified antigens and biodegradable PLGA particles that has not been described before.

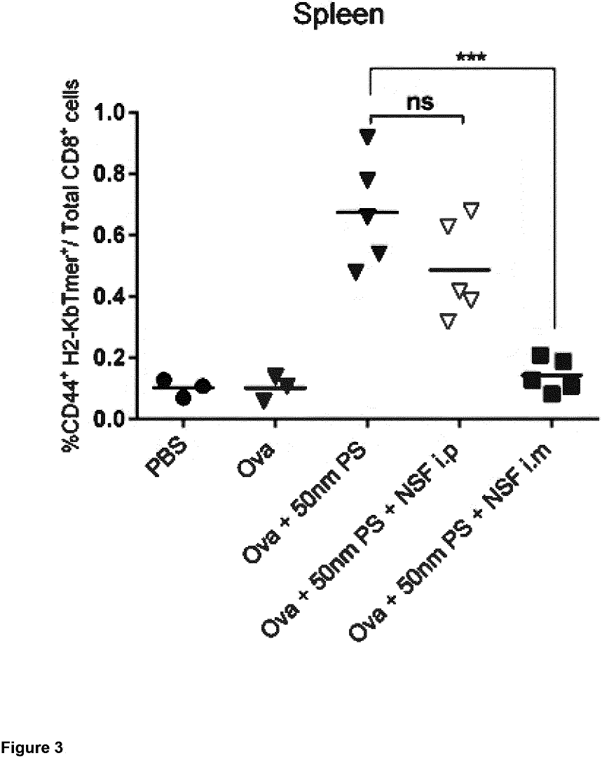

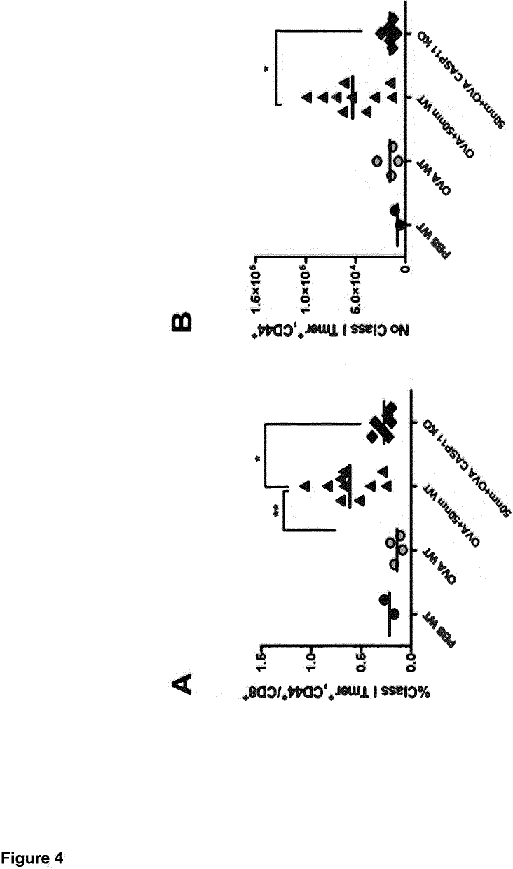

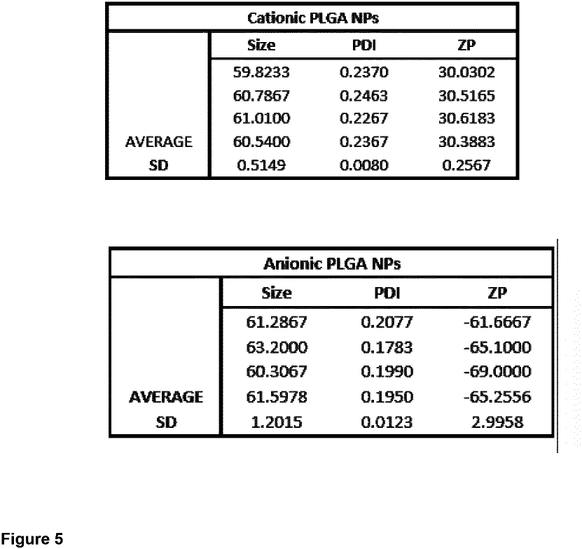

The current inventors have also shown the ability of the 50nm nanoparticles of the invention to induce CD8 responses is dependent on a pathway associated with pyroptosis (programmed immunogenic cell death). This is illustrated in FIG. 3 and FIG. 4. The inventors provide a novel mechanism for adjuvant induced cellular immunity.

The current inventors have surprisingly found that the ability of the particles to induce a type of cell death called pyroptosis is key to their ability to promote CD8 responses.

### Exemplification

The invention will now be described with reference to specific Examples. These are merely exemplary and for illustrative purposes only: they are not intended to be limiting in any way to the scope of the monopoly claimed or to the invention described. These examples constitute the best mode currently contemplated for practicing the invention.

### EXAMPLES

### Example 1

### 50Nm PLGA Nanoparticles Promote CD8⁺ T Cell Responses and IFN-_{Y} when Administered with a Protein Antigen

### Materials and Methods

C57BL/6 mice were immunized on days 0 and 14 with 2mg of particles (50nm) and 20µg of ovalbumin (OVA) via intramuscular route. On day 21 the splenocytes were analysed by flow cytometry to quantify the percentage (FIG. 1A) and number (FIG. 1B) of CD⁺8 T cells specific to the H-2 Kb OVA epitope SIINFEKL using tetramers and IFN-_{Y} secretion was evaluated in splenocytes after re-stimulation with the antigen (FIG. 1C).

Other mice were immunised i.m. on days 1 and 14 with 1mg of biocompatible non-degradable polystyrene particles with positive or negative charge (as per previous derivatization with carboxylic or amine groups) mixed with 10µg of the antigen OVA-NP311 (which is a fusion protein consisting of ovalbumin and the influenza nucleoprotein peptide NP311-325). On day 21 the splenocytes were analysed by flow cytometry to quantify the percentage of CD⁺8 T cells specific to the H-2 Kb OVA epitope SIINFEKL using tetramers (FIG. 1D).

### Results and Conclusion

Results are shown as mean±SEM and symbols represent individual animals. FIG. 1(C) illustrates IFN-γ expression analyzed by ELISA after 72h re-stimulation with the antigen.

These results show that PLGA particles of the current invention are potent inducers of CD8 T cells and the cytokine IFN-_{Y} (which is the hallmark of Th1 responses) (FIGS. 1A, B and 1C).

These results show that nanoparticles can induce an antigen specific CD8 response independently of the charge or degradability of the polymer (FIG. 1D).

### Example 2

### Bigger PLGA Nanoparticles (100nm and 500nm) Fail to Induce CD8⁺ T Cell Responses and IFN-_{Y} when Administered with a Protein Antigen.

### Materials and Methods

C57BL/6 mice were immunized on days 0 and 14 with particles (100nm and 500nm) and ovalbumin (OVA) via intramuscular route. On day 21 the splenocytes were analysed by flow cytometry to quantify the percentage (A) and number (B) of CD8⁺ T cells specific to the H-2 Kb OVA epitope SIINFEKL using tetramers.

### Results and Conclusion

Results are shown as mean±SEM and symbols represent individual animals. FIG. 2(C) illustrates IFN-γ expression analysed by ELISA after 72h re-stimulation with the antigen. Polystyrene nanoparticles were used as a positive control (+ Ctrl).

The response illustrated by FIG. 1 using the particles of the current invention cannot be driven by other PLGA particles of bigger sizes (FIGS. 2 A-B).

### Example 3

### 50Nm Nanoparticles Induce Antigen-Specific CD8 T Cell Responses Through a Pyroptosis Dependent Pathway.

### Materials and Methods

Mice were immunized via the intramuscular (i.m.) route on day 0 and 14 with OVA alone or with 50nm particles. Mice were treated with 250µg of necrosulfonamide (NSF) to inhibit pyroptosis by the intraperitoneal (i.p.) or i.m. route during priming and booster. On day 21, flow cytometry was used to determine the percentage of splenic (FIG. 3) CD8⁺ T cells specific to the H-2 Kb OVA epitope

SIINFEKL (SEQ ID NO. 1)

using tetramers.

### Results and Conclusion

Results are shown as mean±SEM and symbols represent individual animals. Asterisks denote statistical differences calculated as per multiple comparisons ANOVA and Dunnett’s multiple comparison test to determine differences compared to OVA + 50nm PS.

The current inventors have shown the ability of these small PLGA nanoparticles to induce CD8 responses is dependent on necroptosis (FIG. 3). FIG. 4 shows that 50nm particles require Caspase 11, a protein involved in pyroptosis to drive the CD8 response.

### Example 4

### Preparation of Anionic Polymer Nanoparticles of 50 nm Particle Size and Particle Size Calculation.

Materials and Methods

| | |
|---|---|
| Material | Supplier |
| PLGA 50:50 | Sigma Aldrich |
| Phosphatidylserine (brain PS) | Lipoid |
| Tris buffer pH 7.410 mM | Oxoid |
| DMSO | Fisher Scientific |

### Nanoparticle Formulation

### Preparation of Lipid and Polymer Stocks and Buffer

STEP 1. Lipid and polymer were weighed out together and dissolved in DMSO. Heat and bath sonication may optionally be used to achieve completely dissolution of the substances if necessary.

The final concentration of the polymer and lipid was 0.5 mg/mL total, therefore 0.25 mg/mL of PLGA and 0.25 mg/mL PS.

Weight to weight ratio 1:1

STEP 3. Tris buffer is prepared at 10 mM and pH 7.4. NOTE: pH is adjusted using 0.1 M HCI

STEP 4. Buffer is filtrated using a 0.22 µm filter to avoid any impurity or contamination.

### Microfluidics

STEP 5. The desired final concentration of the polymeric NPs is 20 mg/mL, therefore, 160 mL of particles are produced using the Nanoassemblr™ (Precision Nanosystems, Inc.) at a Flow Rate Ratio 1:1 and a Total Flow Rate 10 mL/min. Initial and final waste volumes are 0.35 and 0.05 respectively.

### Tangential Flow Filtration (TFF)

STEP 6. In order to remove the solvent and to concentrate the particles to the desired concentration, Tangential Flow Filtration (Spectrum Labs) is used. The 160 mL of polymeric NPs are concentrated down to 2 mL using TFF, and the solvent is washed with 24 mL of Tris buffer 10 mM, pH 7,4 (12 washes).

STEP 7. Measure size, PDI and ZP of the obtained particles. If the results are within the desired range, particles are ready.

NOTE: If the particles are larger than expected (since the desired particle range is very narrow), particles can be bath sonicated using the Bioruptor for 90 seconds (1 cycle).

### Analytical Techniques

### Measurement of the Particle Size, Size Distribution and Zeta Potential Using Malvern ZS

The particle size of the polymer nanoparticles was determined by dynamic light scattering (DLS) using a Malvern nano ZS (Malvern Instruments, Worcestershire, UK). Three measurements at 25° C. were conducted on the samples, which were previously diluted (10-fold dilution) in Tris buffer pH 7.410 mM and filtered using a 0.22 µm to achieve the optimal particle concentration with the optimum attenuator number (att. 7 - 9). Square single-use plastic cuvettes were filled in with 1 mL of sample and were placed into the instrument which uses a 4-mW He-Ne 633 nm laser to analyse the samples. For zeta potential samples were diluted in the same fashion as for the size and a cell capillary electrophoresis cuvette was used. For collection and data analysis Malvern Dispersion Technology Software (DTS) v.7.12 (Malvern Instruments, Worcestershire, UK) was used.

### Definition of Key Product Parameters (KPP) of a Liquid Product

To ensure the product can be robustly defined with reference to its physico-chemical characteristics your formulation HSPC:Chol:DSPE-PEG2000:DOX will be characterised using the following methods:

| | | |
|---|---|---|
| Assay | Comment | Specification |
| 1. Appearance | Quality test | |
| 2. Particle size & particle size distribution | Vesicle size and size distribution will be measured using photon correlation spectroscopy and laser diffraction as appropriate) | Size: 50-60 nm PDI: 0.15 - 0.25 |
| 3. Particle surface charge | The zeta potential will be measured to give an indication of bilayer composition | Highly anionic Zeta Potential: -65 mV |

### Example 5

Preparation of cationic polymer nanoparticles of 50 nm particle size

| | |
|---|---|
| Material | Supplier |
| Poly(lactic-co-glycolic acid) (PLGA) 50:50 Mw: 30,000 - 60,000 | Sigma Aldrich |
| Dimethyldioctadecylammonium (Bromide Salt) (DDA) | Avanti Polar Lipids |
| Tris buffer pH 7.410 mM | Oxoid |
| Chloroform:Methanol | Fisher Scientific |

### Nanoparticle Formulation

### Preparation of Lipid and Polymer Stocks and Buffer

STEP 1. Individual lipid and polymer are dissolved in a mixture of chloroform: methanol (9:1 v/v). The stock concentrations prepared are:
PLGA 50:50: 20 mg/mL in chloroform: methanol (9:1 v/v)
DDA: 20 mg/mL in chloroform: methanol (9:1 v/v)

STEP 2. Both stocks are mixed together and poured into a round bottom flask
Weight to weight ratio 1:1
Final concentration: 20 mg/mL
Final Volume: 2 mL

STEP 3. Tris buffer is prepared at 10 mM and pH 7.4.

NOTE: pH is adjusted using 0.1 M HCl

STEP 4. Buffer is filtrated using a 0.22 µm filter to avoid any impurity or contamination.

### Manufacture of Cationic Polymeric Formulation Using Lipid Film and Bath Sonicator

STEP 5. Evaporate the organic solvent using a rotary evaporator during 15 min at 200 rpm

STEP 6. Rehydrate the film using 2 mL of filtered Tris buffer 10 mM (pH 7.4) until complete dissolution

STEP 7. Transfer the cationic polymeric particles to a 15 mL falcon tube and place it into a Bioruptor® Plus sonicator (Diagenode, Liege Science Park, 3 Rue bois Saint-Jean, 4102 Ougree, Belgium)

This system uses ultrasounds derived from magnets placed below the water tank and indirectly transfers ultrasonic energy to samples.

STEP 8. Run a cycle of: 90 s cycle (x 10 times) with a delay of 30 s between cycles

### Analytical Techniques

STEP 9. Measurement of the particle size, size distribution and zeta potential using Malvern ZS

The particle size of the polymer nanoparticles was determined by dynamic light scattering (DLS) using a Malvern nano ZS (Malvern Instruments, Worcestershire, UK). Three measurements at 25° C. were conducted on the samples, which were previously diluted (10-fold dilution) in Tris buffer pH 7.410 mM and filtered using a 0.22 µm to achieve the optimal particle concentration with the optimum attenuator number (att. 6 - 7). Square single-use plastic cuvettes were filled in with 1 mL of the diluted sample and were placed into the instrument which uses a 4-mW He-Ne 633 nm laser to analyse the samples. For zeta potential samples were diluted in the same fashion as for the size and a cell capillary electrophoresis cuvette was used. For collection and data analysis Malvern Dispersion Technology Software (DTS) v.7.12 (Malvern Instruments, Worcestershire, UK) was used.

Definition of Key Product Parameters (KPP) of a liquid product

| | | |
|---|---|---|
| Assay | Comment | Specification |
| 1. Appearance | Quality test | |
| 2. Particle size & particle size distribution | Vesicle size and size distribution will be measured using photon correlation spectroscopy and laser diffraction as appropriate) | Size: ~55-65 nm PDI: 0.15 - 0.25 |
| 3. Particle surface charge | The zeta potential will be measured to give an indication of bilayer composition | Highly cationic Zeta Potential: +70 mV |

### Example 6

### Antigen Specific Anti-Tumour Immunity in Mice

### Materials and Methods

Mice were vaccinated on days 0 and 7 with PBS, OVA peptide (10µg) alone, or 50nm PS particles (1mg) plus OVA peptide (10µg), followed by s.c challenge of 3.5x10⁵ B16-OVA tumour cells.

### Results and Conclusion

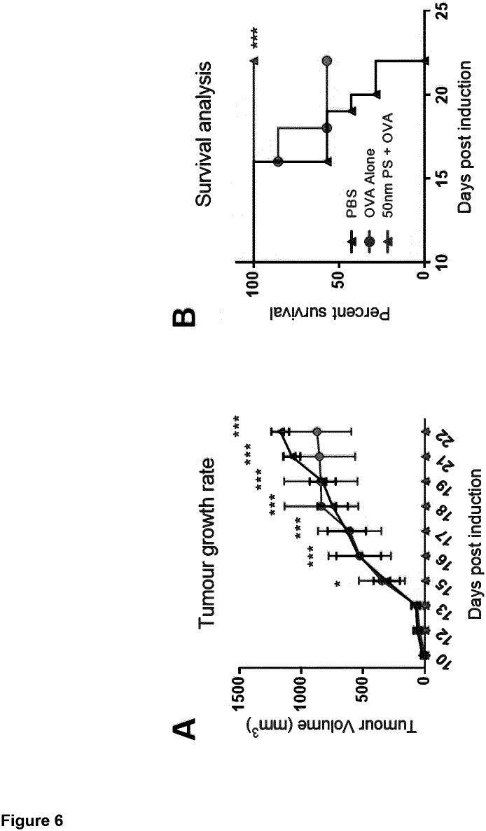

As shown in FIGS. 6A and B a potent antigen specific anti-tumour immunity was driven by 50nm PS nanoparticles. FIG. 6A shows no tumour growth 22 days post induction and FIG. 6B shows 100% survival 20 days post induction.

### Example 7

### Particle Size Does Not Affect Magnitude or Neutralizing Ability of Antibodies but Influences Isotype Class-Switching.

The success of traditional prophylactic vaccines mostly relies on the induction of lasting antibody responses to T-cell dependent epitopes. As a result, vaccine design and adjuvant discovery have used antibody titers and neutralizing activity as the main correlates of vaccine efficacy and adjuvanticity.

Therefore, the inventors evaluated how particle size impacts the magnitude and quality of antibody responses. Polymeric polystyrene (PS) particles are biocompatible particles with intrinsic adjuvant properties which allow greater control of particle size compared to other biodegradable polymers including PLGA. Therefore, PS particles ranging from 50nm to 100µm in diameter were selected as model particulate adjuvants.

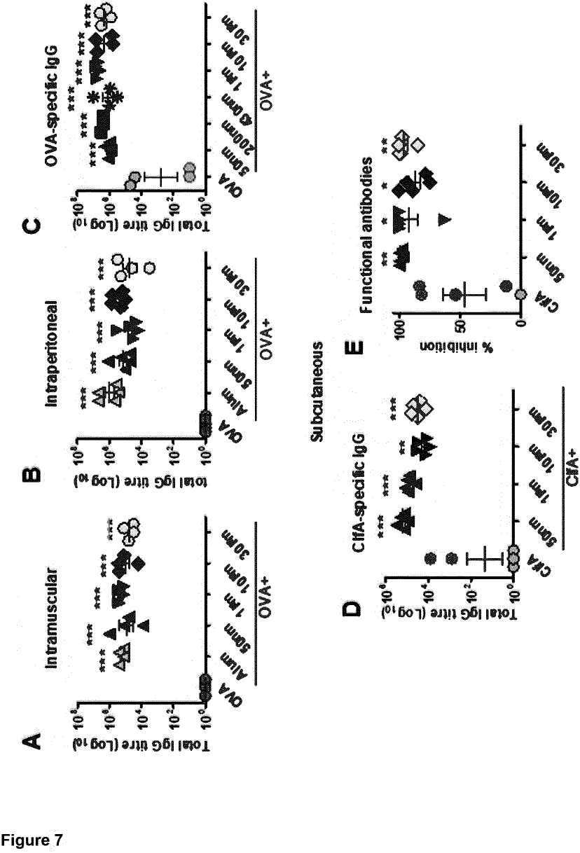

PS particles were mixed with OVA at pre-established antigen/particle ratios optimized for intramuscular (i.m.), intraperitoneal (i.p.) or subcutaneous (s.c.) administration. Mice were vaccinated with vehicle, OVA alone or PS+OVA on days 0 and 14 and OVA-specific IgG responses were evaluated in serum on day 21. All particle sizes 50nm-30µm elicited a strong OVA-specific IgG response although larger 100µm particles given i.p. where marginally less efficient. PS particles induced responses comparable in magnitude to the OVA-specific IgG titers induced by the gold standard adjuvant alum via both i.m. (FIG. 7A) and i.p. routes (FIG. 7B). The inventors previously showed that PLGA particles in the 400nm range induced antigen specific antibodies when given s.c.. In order to determine if PS particles could elicit similar responses, mice were vaccinated with PS+OVA s.c. as per the scheme above and additional sizes in the nano-range (200nm and 430nm) were also tested. Again, all particle sizes induced robust OVA-specific IgG after s.c. vaccination including all those in the nanometer range (FIG. 7C). Finally, to establish whether particles could induce a protective humoral response to a clinically relevant antigen, mice were vaccinated s.c. with recombinant Staphylococcal Clumping Factor A (ClfA) admixed with particles. ClfA is a surface protein and virulence factor of Staphylococcus aureus that binds to fibrinogen and fibrin, promoting bacterial adhesion to blood clots in vivo and ex vivo to biomaterials coated in plasma proteins. Adhesion of the bacteria can be blocked by neutralizing antibodies against ClfA which allowed the inventors to determine the functionality of the antibodies induced after immunization with the particles by measuring the capacity of the serum of vaccinated mice to inhibit S. aureus adhesion to immobilized fibrinogen in vitro. Particles in all size range induced ClfA IgG (FIG. 7D) and showed comparable neutralizing activity (FIG. 7E).

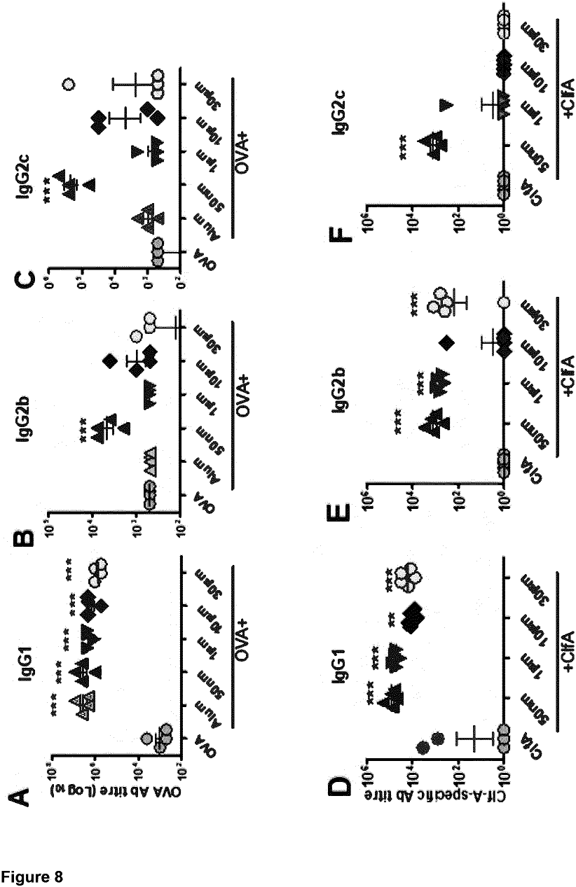

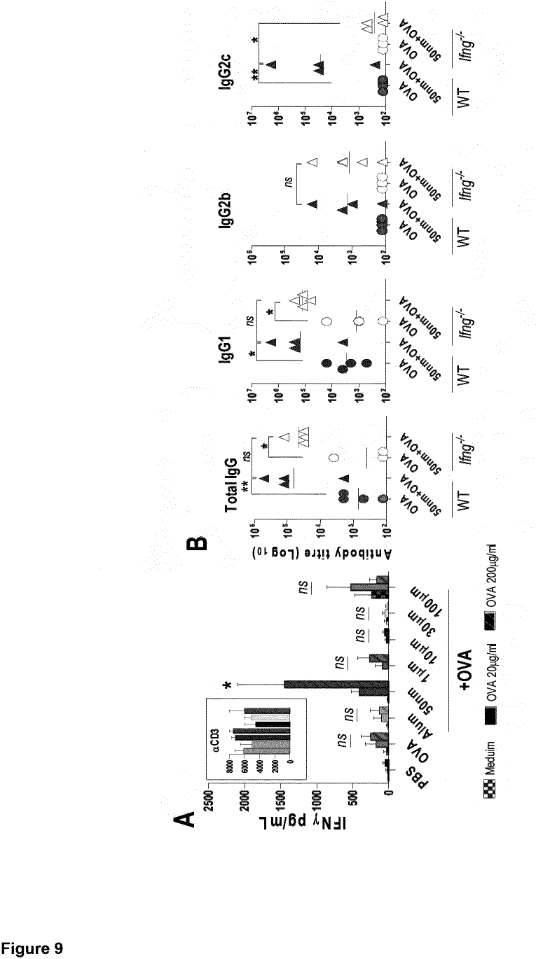

Finally, the inventors assessed the quality of humoral responses by analyzing antibody isotypes. All particle sizes tested induced comparable IgG1 titers both i.m. (FIG. 8A) and s.c. (FIG. 8D), and many particle sizes induced IgG2b when given s.c. with ClfA, (FIG. 8E). Notably, only 50nm particles induced class switching to IgG2c after i.m. vaccination with OVA (FIG. 8C) or s.c. vaccination with ClfA (FIG. 8F). In sum, these data indicate that while the overall magnitude of the humoral response and functionality of the antibodies is independent of particle size, particle diameter determines the quality of antibody responses by regulating class-switching with 50nm particles presenting a unique ability to selectively induce antigen-specific IgG2c. IgG2c responses require IFN. Therefore, the selective enhancement of IgG2c supports the inventors finding that the particles are effective in promoting Th1 response.

### Example 8

### Size-Dependent Induction of Antigen-Specific IFN-γ Drives IgG2c Class-Switching

Antibody isotype switching is influenced by T lymphocyte-derived cytokines. In rodents IL-4 promotes switching to IgG1 and IgE, whereas IFN-_{Y} stimulates IgG2c/IgG2a in C57/BL6 or BALB/c mice respectively. Previously the inventors showed that 50nm polystyrene particles combined with OVA or the tuberculosis candidate antigen H56 had superior ability to induce Th1 responses compared to alum. The hypothesized that this unique ability of small nanoparticles to promote IgG2c class-switching was linked to their superior capacity for driving IFN-_{Y} secretion compared to larger particles. To test this hypothesis, cytokine secretion was assessed upon antigen re-stimulation of splenocytes ex vivo, obtained from mice previously vaccinated i.m. with OVA and different PS sizes. Splenocytes from animals vaccinated with 50nm PS particles were the only ones to significantly upregulate IFN-_{Y} secretion after OVA compared to those obtained from mice vaccinated with the antigen alone or combined with any other particle size (FIG. 9A). Vaccination of IFN-_{Y} knockout (lfng^{-/-}) mice with 50nm PS+OVA failed to induce IgG2c, confirming that IFN-_{Y} is essential for IgG2c switching, whereas IgG, IgG1 and IgG2b levels remained comparable in lfng^{-/-} and wild-type (WT) mice (FIG. 9B). These results confirm that 50nm particles promote IFN-_{Y}-driven antibody class-switching towards IgG2c.

### Example 9

### Particle Diameter Determines the Induction of Durable Antigen-Specific CD8⁺ and CD4⁺ T Cell Responses Independently From Vaccination Route and Confer Protection Against Melanoma

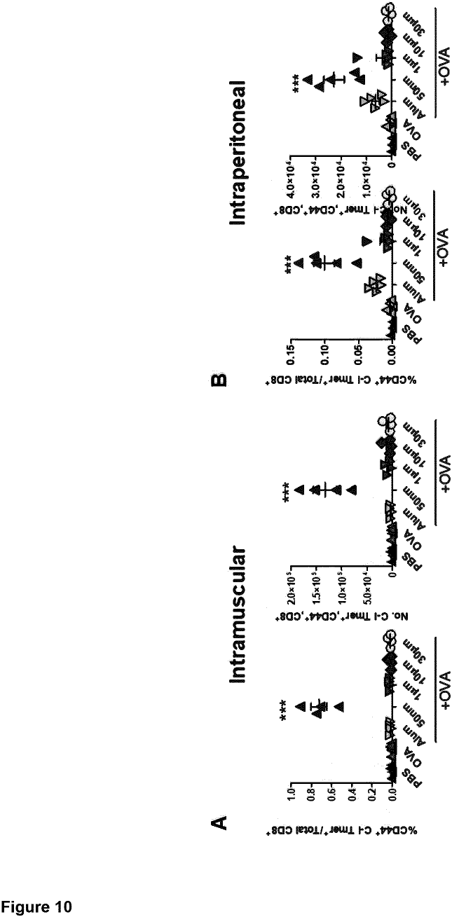

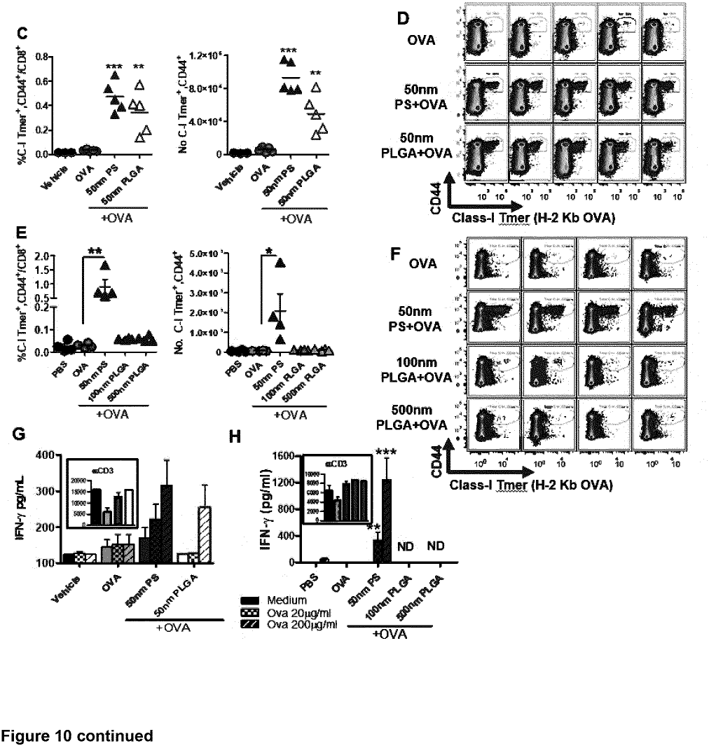

Intrinsic particle characteristics such as size, geometry and hydrophobicity influence the induction of CTL and IFN-_{Y} responses, but other factors such as the immunization route also play an important role. By primarily affecting the kinetics of antigen and adjuvant transport to the lymph nodes, these factors can impact the DCs subsets being targeted and therefore the efficiency of CD8⁺ T cell cross-priming. Small nanoparticles (>200nm) are taken up by locally at the injection site but can also freely reach the nodes in short times after injection directly target resident DCs including CD8α⁺ DCs and plasmacytoid DC (pDCs) that are specialized in cross-presentation of antigens and priming of CD8⁺ T cells. In contrast, particles <200nm are more likely to be taken up by resident cells at the injection site which ferry them to the lymph nodes. While small nano-sized particles can efficiently prime CTL responses, the relative contribution of particle intrinsic attributes i.e. size vs influence of immunization strategies on the induction of CD8⁺ T cell responses by particulate formulations has not been fully resolved. To address whether particle size can drive CTLs independently from vaccination route, the inventors assessed antigen-specific CD8⁺ T cell responses after immunization with admixed OVA and particles of different diameters, administered either i.p. or i.m. using the same prime boost regime as before. Seven days after booster, antigen-specific CD8⁺ T cell responses were quantified using tetramers for the H-2 Kb OVA epitope SIINFEKL by flow cytometry. Remarkably, only 50nm particles induced a significant increased antigen-specific CD8⁺ T cells in spleens of animals vaccinated either i.p. or i.m. (FIGS. 10A-B), suggesting that size rather than administration route is key to priming of CD8⁺ T cell responses by polymeric particles.

To investigate if particle composition influences this response, antigen-specific CD8⁺ T cells were also compared in mice receiving biocompatible OVA alone or in combination with 50nm PS particles or biodegradable PLGA particles of 50-60nm, 100nm and 500nm in diameter. Importantly, only 50nm PLGA particles were able to induce a significant number of antigen-specific CD8⁺ T cell responses after i.m. vaccination comparable to the CTL response elicited by 50nm PS particles (FIGS. 10C-D). In contrast, 100nm and 500nm particles failed at stimulating T CD8⁺ responses (FIGS. 10E-F). Likewise, only splenocytes from animals vaccinated with 50nm PLGA particles induced antigen-specific IFN-_{Y} production comparable to those from mice vaccinated with 50nm PS particles (FIG. 10G) when stimulated ex vivo with the antigen. Whereas IFN-_{Y} was not detected in splenocytes isolated from animals immunized with antigen and 100nm or 500nm PLGA (FIG. 10H) indicating that size rather than polymer composition determines the induction of Th1 responses.

To determine whether size also influenced CD4⁺ T cell responses, mice were vaccinated with the influenza A nucleocapsid protein peptide

(SEQ ID NO. 2) QVYSLIRPNENPAHK

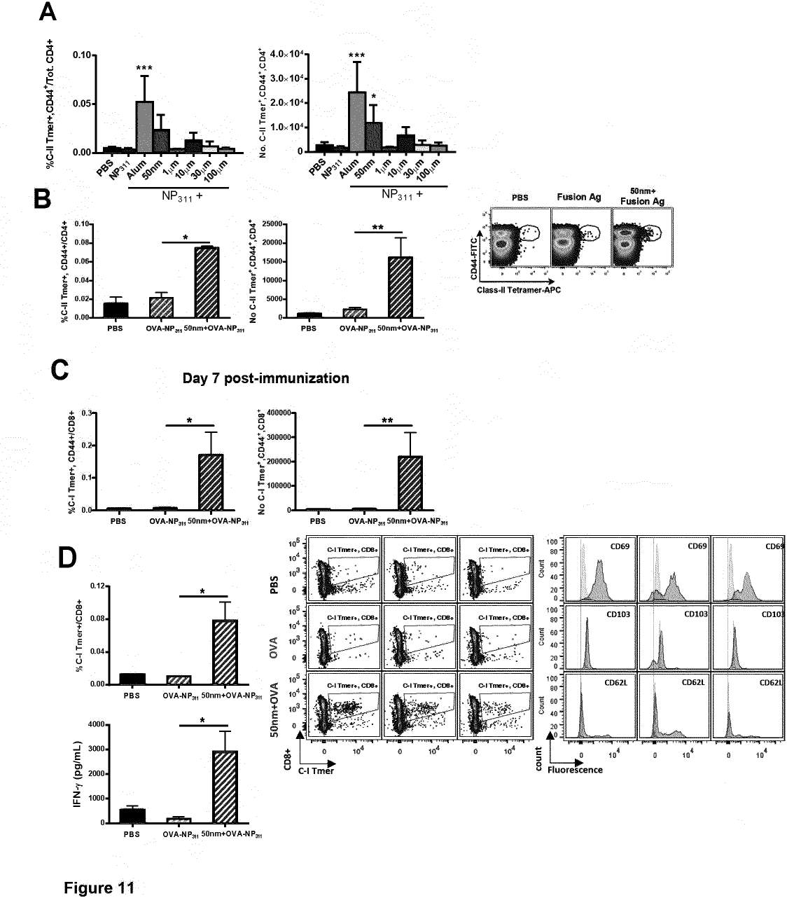

(NP₃₁₁₋ ₃₂₅) alone or in combination with particles. The use of NP₃₁₁₋₃₂₅, a known class-II restricted epitope in C57BL/6 mice, allowed us to overcome the poor performance of OVA I-A(b) tetramers as suitable MHC-II tetramers for NP₃₁₁₋₃₂₅ are available. Alum was used as a positive control and as predicted (McKee et al, International Immunology, 20, 659-669, 2008) it induced the highest increase in antigen-specific CD4⁺ T cells on day 21. Fifty nm PS particles followed alum in their capacity to induce Ag-specific CD4+ T cell responses, while none of the other sizes tested induced a significant response compared to the antigen alone (FIG. 11 A). To confirm that 50nm particles were able to induce CD4⁺ T cell responses when mixed with a protein in addition to a peptide, and via i.m. route, mice were vaccinated i.m. with a fusion antigen generated by chemical conjugation of OVA and NP₃₁₁₋₃₂₅ (NP₃₁₁-OVA) admixed with 50nm particles or alone. Seven days after booster, the number of NP₃₁₁₋₃₂₅ specific CD4⁺ T cells in spleens was enhanced in mice that received 50nm PS + NP₃₁₁-OVA (FIG. 11B). As expected, these animals also upregulated SIINFEKL class-l restricted CD8⁺ T cells (FIG. 11C). The breadth of antigen-specific T cell responses induced by 50nm particles was assessed 100 days after booster. Class I SIINFEKL specific CD8⁺ T cells were still upregulated in animals vaccinated with NP₃₁₁-OVA+50nm particles as opposed to those that received antigen alone (FIG. 11D, upper left panel and dot plots). These cells were characterized by the upregulation of the canonical memory marker CD69 and lack of CD62L expression and CD103 (FIG. 11D, histograms). Previously, we determined that after i.m. vaccination with 50nm PS particles CD4⁺ T cells are the main producers of antigen-specific IFN-_{Y}. Restimulation of splenocytes with NP₃₁₁-OVA on day 100 after booster significantly enhanced the secretion of IFN-_{Y} in mice immunized with 50nm PS + NP₃₁₁-OVA compared to antigen alone, suggesting the presence of functional Ag-specific CD4⁺ memory T cells (FIG. 11D, lower left panel).

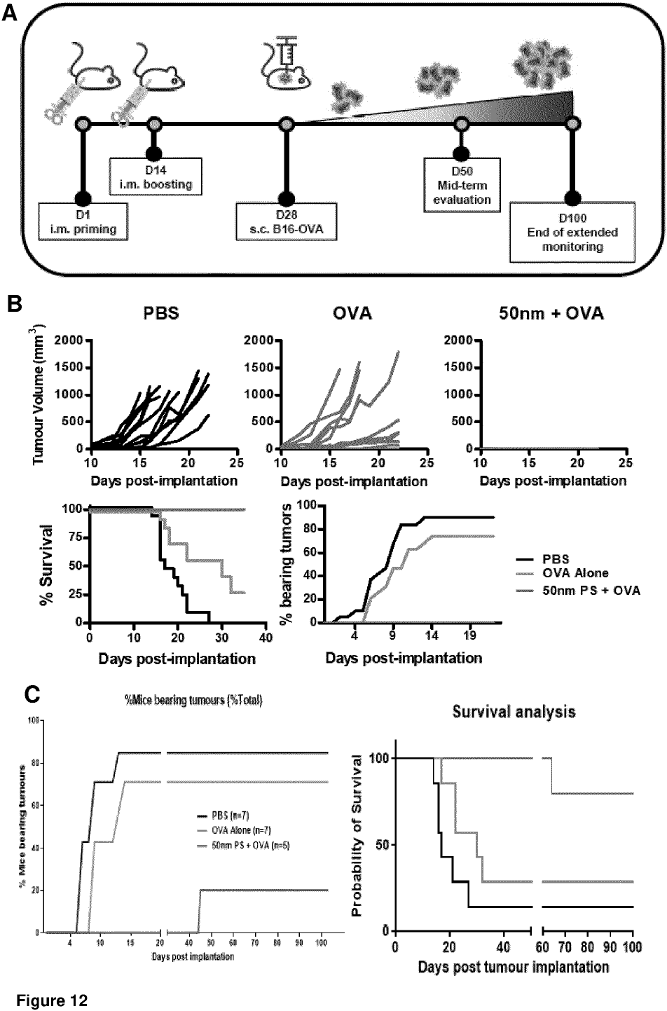

To further assess the functionality of the response elicited by vaccination with nanoparticles, the inventors evaluated their ability to confer prophylactic protection against the B16-F10 melanoma model using cancer cells genetically engineered to express the vaccine antigen OVA (B16-OVA). This model was selected as protection is known to strongly rely on IFN-_{Y}, and antigen-specific CD8⁺ and CD4⁺ T cells. Fourteen days after an i.m. booster vaccination, B16-OVA cells were implanted s.c. and tumor growth was monitored and recorded daily (FIG. 12A). Mid-term monitoring showed that mice vaccinated with 50nm PS particles and OVA were fully protected against the melanoma challenge, whereas all animals in the mock group (PBS) developed tumors and died by day 25 post-implantation. Although vaccination with OVA alone delayed the development of tumors compared to PBS, 80% of the animals still developed tumors within two weeks of implantation and 75% succumbed to challenge by day 35 (FIG. 12B). Extended analysis for 100 days after implantation of the tumors showed that vaccination with 50nm PS+OVA conferred long-lasting protective immunity against the B16 melanoma (FIG. 12C).

Altogether our results reveal that size critically determines the ability of polymeric particles to induce antigen-specific Th1 and CD8⁺ T cell responses and identify 50nm as the optimal size for promoting long-lasting and durable CMI after vaccination. CMI induced by 50nm particles is independent of the immunization route and polymer composition as biodegradable PLGA particles elicited a comparable response.

### Example 10

### IL-1 and IL-18 Are Respectively Required for IFN-_{Y} and CD8⁺ T Cell Responses Induced by Nanoparticles

The cytokines of the IL-1 family importantly modulate priming, expansion and survival of T cells either by promoting maturation of antigen presenting cells or directly on lymphocytes.

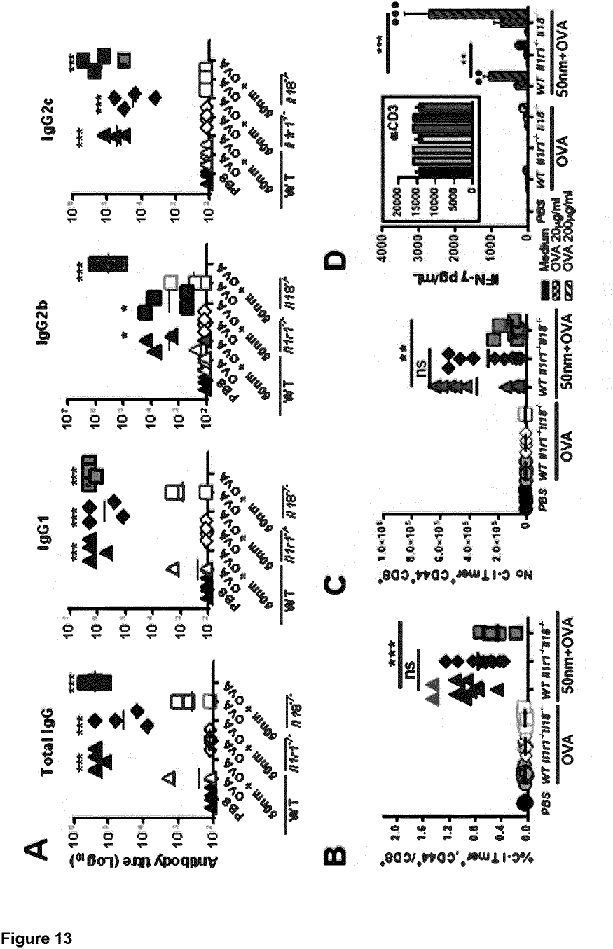

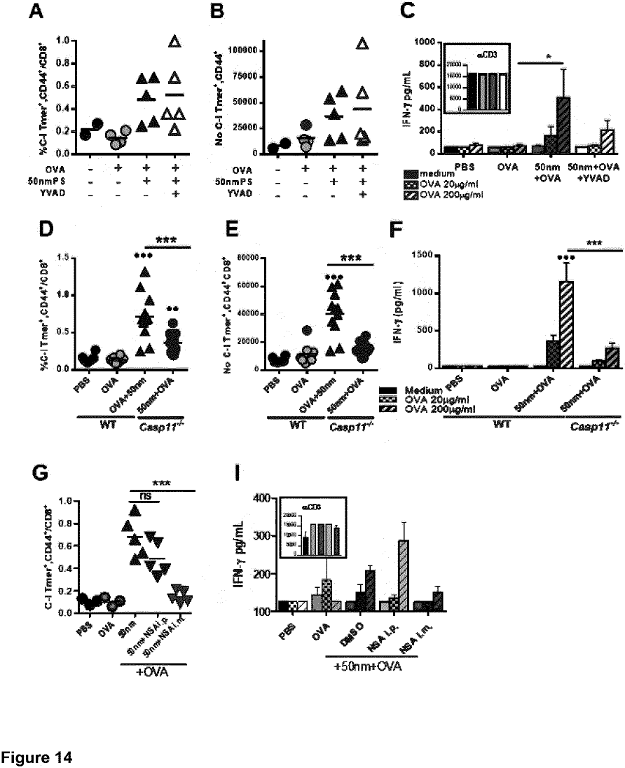

Vaccination of mice deficient in IL-1R1 and IL-18 with 50nm particles and OVA showed that neither cytokine is required for the induction of OVA-specific IgG responses. Overall, the magnitude and IgG isotype profiles were similar in both KO strains and WT mice with the exception of a IgG2b production which showed a trend towards increased titres in II18^{-/-} mice (FIG. 13A). In contrast, the analysis of OVA H-2Kb-restricted CD8⁺ T cells revealed that IL-18 is required for the CTL responses induced by 50nm particles, as both the percentage and total number of antigen-specific CD8⁺ T cells in spleens were significantly reduced in II18^{-/-} mice compared to wildtype (WT) animals. Intriguingly, despite reports suggesting that IL-1 can directly support expansion and effector function of CD8⁺ T cells (Ben-Sasson et al., Journal of experimental medicine, 210, 491-502, 2013a; Ben-Sasson et al.,Cold Spring Harbour symposia on quantitative biology 78, 117-124, 2013b), OVA-specific CD8⁺ T cell numbers were unaffected by impaired IL-1 signaling (FIGS. 13B-C). Comparison among mice vaccinated with 50nm+OVA, revealed that II1r1^{-/-} consistently produced significantly less IFN-_{Y} than WT when stimulated with OVA, whereas II18^{-/-} mice revealed an unexpected but consistent significant increase of IFN-_{Y} secretion compared to WT (FIG. 13D).

### Example 11

### The Non-Canonical Inflammasome Sensor Caspase-11 and the Pyroptosis Effector GSDMD are Essential Mediators of Nanoparticle-Induced Cell Mediated Immunity

The protease caspase-1 (casp-1) controls the secretion of leaderless proteins including L-18 and IL-1β. Upstream, sensing of varied infectious and non-infectious stimuli including PAMPs, particulate matter, ion flux or mitochondrial dysfunction and redox stress, trigger the assembly of inflammasomes which recruit pro-caspase-1 leading to autoproteolysis and activation. In addition to casp-1 canonical inflammasomes, non-canonical inflammasomes using the murine caspase-11 (or human caspases ⅘) or caspase-8 can drive secretion of leaderless proteins including IL-1 and IL-18. Therefore, the inventors sought to determine whether caspase 1 or 11 were contributing to antigen-specific CMI induced by 50nm polymeric particles. To evaluate this, the inventors pharmacologically inhibited casp-1 by locally injecting Ac-YVAD-fmk at the time of vaccination. In parallel, casp-11 deficient mice (Casp11^{-/-}) were also vaccinated.

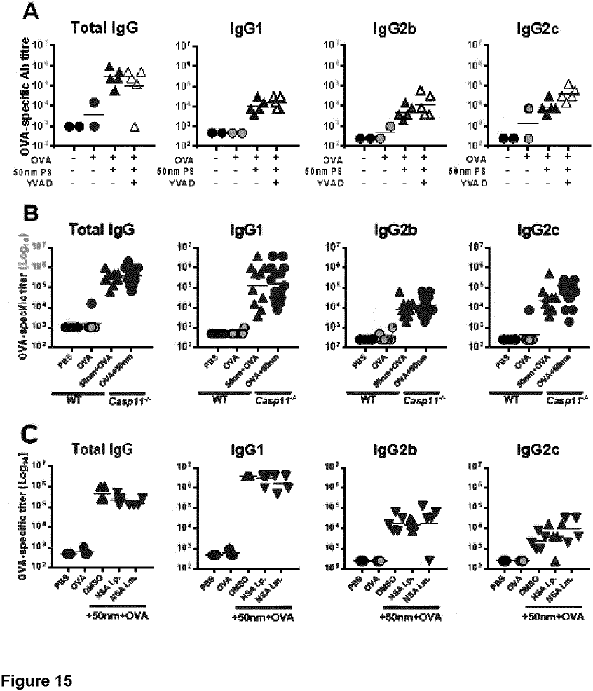

Pharmacological inhibition of casp-1 did not significantly alter the induction of antigen-specific CD8⁺ T cells compared to WT mice (FIGS. 14A, B). However, antigen restimulation of splenocytes from animals Ac-YVAD-fmk treated animals showed a trend towards decreased IFN-_{Y} secretion (FIG. 14C). Inhibition of casp-1 did not alter antibody responses in magnitude or quality (FIG. 15A). On the other hand, casp-11 deficiency significantly impaired particle-induced antigen-specific CD8⁺ T cell responses both in terms of percentage of SIINFEKL specific CTLs and total numbers in spleen, compared to WT mice that received the particles (FIGS. 13D, E). Deficiency in casp-11 also caused a 4-fold decrease in IFN-_{Y} secretion upon antigen stimulation compared to WT splenocytes (FIG. 13F). As before, casp-11 deficiency did not affect antibody responses (FIG. 15B).

Activation of canonical capsase-1 inflammasomes or caspase-11 non-canonical inflammasomes leads to downstream cleavage of GSDMD. Cleaved GSDMD N-domains interact with acidic phospholipids on the inner side of the cell membrane and oligomerize forming transmembrane pores. The GSDMD pores allow the release of IL-1β and IL-18 as well as pyroptotic cell death. However, depending of the activation context cytokine release can occur during or independently of cell death when cells adopt a state of hyperactivation.

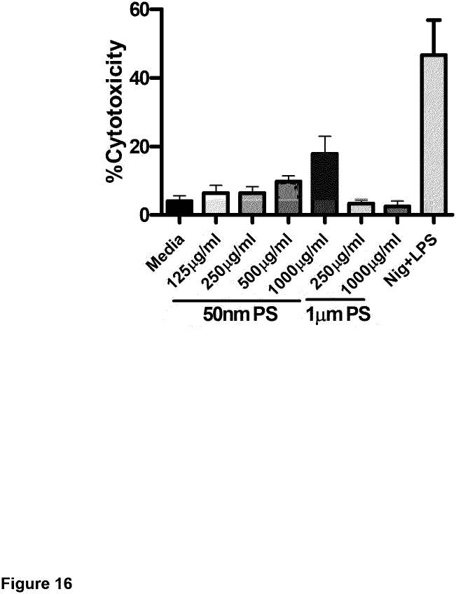

The inventors hypothesized that particles could act by directly targeting skeletal muscle cells inducing local death that would in turn release danger-associated molecular patterns that can act as endogenous adjuvants to potentiate the immune response. In vitro data 50nm particles induced cell death in differentiated murine myotubes as per LDH release. In contrast 1 micron particles did not (FIG. 16).

The inventors sought to investigate if GSDMD was required in vivo for particle induced CMI and humoral responses. The inhibitor necrosulfonamide (NSA) binds to GSDMD C191 residue impeding p30-GSDMD oligomerization. As a result NSA selectively blocks pyroptosis and IL-1β release in human and murine cells without affecting TLR signaling, inflammasome formation or gasdermin E-mediated cell death, and while NSA inhibits the necroptosis effector mixed lineage kinase domain like (MLKL) protein in humans, this effect is not found in murine cells. In order to assess the relevance of local vs systemic GSDMD activation in particle-induced responses, mice were administered NSA intraperitoneally 1h prior vaccination or locally in the muscle at the time of immunization. Inhibition of GSDMD had no impact on antibody responses regardless of the administration route of the inhibitor (FIG. 15C). On the other hand, i.p. injection of NSF induced a partial decrease in antigen-specific CD8⁺ T cell that was abolished by local administration of NSA (FIG. 13G). This indicates that local activation of GSDMD is necessary for priming of CTL responses by 50nm particles. In addition, local administration of NSA downregulated IFN-_{Y} responses in ex vivo restimulated splenocytes (FIG. 13I).

Altogether, the data suggest that 50nm polymeric particles trigger local activation of the non-canonical inflammasome sensor caspase-11 and GSDMD promoting pyroptosis of cells at the injection site, secondary caspase-1 activation with the consequent release of active IL-1 and IL-18 to promote antigen-specific IFN-_{Y} and CD8⁺ T cell responses respectively.

### DISCUSSION

Despite the growing number of candidates, polymeric particle adjuvants have not yet been included in any clinically approved vaccine. Two of the most crucial steps in rational adjuvant design are: to define the design principles linking particle characteristics to specific immune profiles; and to elucidate their modes of action. The use of polydisperse formulations with overlapping sizes, different antigen incorporation methods, inclusion of co-adjuvants or varied immunization strategies have led to conflicting results and prevented the identification of an optimal particle size linked to specific types of immune responses.

The inventors used biocompatible and highly monodisperse, endotoxin-free polystyrene particles mixed with protein antigens as a model particulate vaccine to resolve the longstanding question about the role of particle size in adjuvant-driven immunity. Their work showed that particle size differentially regulates discrete components of the immune response, namely humoral and cellular responses. Particle diameter did not significantly impact humoral responses in terms of magnitude or neutralizing capacity of antibodies (FIGS. 7 and 8). However, it critically determined the ability of solid polymeric particles to promote cell-mediated immunity to protein antigens. The inventors unambiguously identified 50±10nm as the optimal size for the induction of CD8⁺ T cell responses and IFN-_{Y} by polymeric nanoparticles (FIG. 9 and FIG. 10), independently of the administration route or the antigen used. In addition, the inventors showed efficacy in the melanoma model as particles given prophylactically with antigens expressed by the cancer cells conferred protection (FIG. 12) and provided evidence of durable CD8⁺ T cell responses (FIG. 11D).

The capacity of 50nm particles to induce CMI extends beyond model polystyrene particles, as the inventors showed that 50nm biodegradable particles made of PLGA also induced antigen-specific CD8⁺ T cell responses and IFN-_{Y} production comparable to PS particles. In contrast, their 100nm and 500nm PLGA counterparts failed to induce CMI (FIGS. 10C-H). PLGA particles are one of the most promising adjuvant candidates as many PLGA particles and other products are approved for human use by the FDA and EMA. While PLGA particles have been effectively used as drug carriers, they yet have to reach the vaccine industry. Generally, the induction of CMI had been poor. This has led to alternative co-formulation strategies with TLR agonists and other co-adjuvants or complex antigen encapsulation or conjugation methods that attempt against formulation stability, increase the cost and challenge their translation into clinically relevant preparations. Prior to the current invention, limitations in the PLGA particle formulation processes restricted their production to sizes between 80nm and 250µm. But new platforms including the microfluidic technology used to manufacture the 50nm particles used in this work, now allow for fine control of particle size, particles with low polydispersity index, avoiding the use of harmful solvents and allowing escalation for mass production of adjuvants. This together with the identification of the optimal particle size able promote CMI, could importantly accelerate the development of PLGA adjuvants for anticancer and antiviral vaccines.

The inventors identified the non-canonical inflammasome sensor caspase-11 and the pyroptosis effector gasdermin D as novel actors in the induction of CMI induced by 50nm particles (FIG. 14). It is important to highlight that a role for caspase-11 in the induction of Th1 responses has been previously reported for GLA-SE. The ability of GLA-SE to engage the non-canonical inflammasome pathway has been attributed to the glucopyranosyl lipid moiety, which may interact with caspase-11 in a similar way to its original ligand LPS. However, the particles and antigens used in this work showed no evidence of LPS contamination as they failed to induce IL-6, TNF-α or IL-1β secretion in unprimed dendritic cells, which are known to produce inflammatory cytokines after sensing LPS concentrations as low as 10pg/ml. Therefore, it is plausible that 50nm particles induce the release of specific DAMPs able to directly engage caspase-11.

The requirement for GSDMD for particle-induced CMI suggests that either a different endogenous casp-11 ligand is induced by the particles or that the outcomes downstream casp-11 activation are cell dependent. In fact, while particles caused limited cell death in DCs when incubated in vitro, they induced lytic cell death and casp-11 activation in skeletal myotubes

Both IL-1 and IL-18 were needed for nanoparticle-induced CMI. The data suggest a division of labor for IL-1 and IL-18 regulating Th1 and CTL responses as IL-1R1 signaling was required for IFN-_{Y} secretion which we showed depends on CD4⁺ T cells, whereas IL-18 mainly contributed to CD8⁺ T cell responses (FIG. 13A). This finding is in agreement with the fact that IFN-_{Y} deficiency did not impact the CD8⁺ T cell numbers elicited by the nanoparticles (FIG. 9B).

While canonical and non-canonical inflammasomes can trigger pyroptosis, cleavage of pro-IL-1β and pro-IL-18 classically require caspase-1 activity. Consequently, blocking casp-1 enzymatic activity and therefore activation of IL-1 and IL18, should have led to abrogation of both IL-1-induced IFN-_{Y} production and IL-18-driven CD8⁺ T cell responses. However, administration of Ac-YVAD-fmk only affected IFN-_{Y} secretion leaving the CD8 response intact.

Interestingly, numerous reports have suggested that caspase-4 inflammasomes (one of the human orthologs for murine casp-11) directly cleaves IL-18 during intestinal infection with Salmonella enterica, whereas caspase-11, but not caspase-1 mediated proteolytic activation of IL-18 in a model of Salmonella infection. Therefore, the current inventors propose that caspase-11 directly mediates activation of IL-18 triggered by 50nm particles.

In summary, the inventors demonstrate that modification of particle size can be used as a strategy to boost the ability of polymeric particulate adjuvants to induce long-lasting and protective T CD8⁺ and Th1 responses. For the first time the inventors demonstrate the involvement of the non-canonical inflammasome sensor caspase-11 and the pyroptotic effector Gasdermin D in the mode of action of particulate adjuvants underscoring the importance of non-canonical inflammasome activation for the induction of cell mediated immunity by nanoparticle vaccines. These results open new avenues for the rational design of anticancer and antiviral vaccines.

1. A polymeric nanoparticle having a diameter of less than 80nm for use in inducing a CD8 response and/or a Th1 response against an immunogenic species in a subject.

2. The polymeric nanoparticle for use of claim 1, wherein the response induced is a CD8 response.

3. The polymeric nanoparticle for use of claim 1, wherein the response induced is a Th1 response.

4. The polymeric nanoparticle for use of claim 1, wherein the response induced is a CD8 response and a Th1 response.

5. The polymeric nanoparticle for use of claim 2, wherein the CD8 response is mediated by caspase 11 and/or gasdermin D.

6. The polymeric nanoparticle for use of claim 3, wherein the Th1 response is mediated by caspase 11 and/or gasdermin D.

7. The polymeric nanoparticle for use of any one of the preceding claims, wherein the response is induced without a co-adjuvant.

8. The polymeric nanoparticle for use of any one of the preceding claims, wherein the polymeric nanoparticle has a diameter of from about 30nm to about 65nm.

9. The polymeric nanoparticle for use of claim 8, wherein the polymeric nanoparticle has a diameter of from about 40nm to about 60nm.

10. The polymeric nanoparticle for use of any one of the preceding claims, wherein the polymeric nanoparticle is formulated as a composition, the composition comprising the nanoparticle and the immunogenic species, wherein the nanoparticle and immunogenic species are mixed in the composition.

11. The polymer nanoparticle for use of claim 10, wherein the immunogenic species is adsorbed to the surface of the nanoparticle and/or unbound in the composition.

12. The polymeric nanoparticle for use of any one of the preceding claims, wherein the nanoparticle is endotoxin free.

13. The polymeric nanoparticle for use of any one of the preceding claims wherein the nanoparticle is biocompatible.

14. The polymeric nanoparticle for use of any one of the preceding claims, wherein the nanoparticle is a solid particle.

15. The polymeric nanoparticle for use of any one of the preceding claims, wherein the nanoparticle is biodegradable.

16. The polymeric nanoparticle for use of any one of the preceding claims wherein the polymer is poly(D, L-lactide-co-glycolide) (PLGA) polylactic acid (PLA) polyphosphazene or chitosan.

17. The polymeric nanoparticle for use of any one of the preceding claims, wherein the immunogenic species is an antigen.

18. A composition comprising an adjuvant and an immunogenic species, wherein the adjuvant comprises a biocompatible and/or biodegradable polymeric nanoparticle having a diameter of about 40nm to about 60nm and wherein the adjuvant and immunogenic species are mixed in the composition.

19. The composition of claim 18, wherein the immunogenic species is adsorbed to the surface of the nanoparticle and/or unbound in the composition.

20. The composition of claim 18 or 19, wherein the nanoparticle is endotoxin free.

21. The composition of any one of claim 18 to 20, wherein the nanoparticle is solid.

22. The composition of any one of claim 18 to 21 which is a vaccine composition.

23. The composition of any one of claims 18 to 22, for use in a method of inducing a CD8 response and/or Th1 response in a subject.

24. The composition of any one of claims 18 to 22, for use in a method of treating or preventing a disease or condition in a subject.

25. The composition for use of claim 24, wherein the disease or condition is a bacterial infection or a viral infection or cancer or a chronic infectious disease.

26. The composition for use of claim 25, wherein the disease is selected from the group comprising SARS-CoV-2, influenza, hepatitis, herpes zoster, dengue, zika, an infection caused by intracellular bacteria or bacteria that require effective activation of macrophages for intracellular killing, an infection caused by mycobacteria, intracellular parasites or parasites with intracellular developmental stages.

27. The vaccine composition of claim 22, for use in vaccine therapy to prevent or treat a condition or disease in a subject.

28. A method of vaccinating a subject comprising administering the composition of any one of claims 18 to 22 to a subject.

29. A method of treating or preventing a disease in a subject, the method comprising administering the composition of any one of claims 18 to 22 to a subject.

30. A method of inducing a CD8 response and/or Th1 response in a subject, the method comprising administering the composition of any one of claims 18 to 22 to a subject.


SEQUENCE LISTING (2 sequences)

SEQ ID NO: 1 - protein, 8 aa, Artificial Sequence
  OVA epitope
SIINFEKL

SEQ ID NO: 2 - protein, 15 aa, Artificial Sequence
  Nucleocapsid protein peptide
QVYSLIRPNENPAHK